(19) United States
(12) Patent Application Publication
(10) Pub. No.: US 2026/0315303 A1
(43) Pub. Date: Oct. 15, 2026

(54) POWERED EXOSKELETONS COMPRISING A HYBRID BRAIDED-COMPOSITE SHEAR INTERFACE

(71) Applicant: Dephy, Inc., Boxborough, MA (US)

(72) Inventors: Luke Mooney, Sudbury, MA (US); William Marvin, Canton, MA (US); Jonathan Cummings, Concord, MA (US); Hugh Herr, Concord, NH (US)

(73) Assignee: Dephy, Inc., Boxborough, MA (US)

(21) Appl. No.: 19/548,283

(22) Filed: Feb. 24, 2026

Related U.S. Application Data
(63) Continuation of application No. PCT/US2024/044058, filed on Aug. 27, 2024.
(60) Provisional application No. 63/534,933, filed on Aug. 28, 2023.

(51) Int. Cl. A61H 3/00; A61H 1/02

(52) U.S. Cl. CPC A61H 3/00; A61H 1/0266; A61H 2003/007; A61H 2201/1642; A61H 2201/169; A61H 2201/5007; A61H 2201/5069; A61H 2201/5084

(57) ABSTRACT

An ankle-foot exoskeleton to augment motion of a user is provided. The ankle-foot exoskeleton can include a braided structure configured to at least partially encapsulate a lower limb of the user, the braided structure configured to extend from at least below a knee of the lower limb of the user to at least above an ankle of the lower limb. The ankle-foot exoskeleton can include one or more actuators mechanically integrated with the braided structure. The ankle-foot exoskeleton can include one or more processors, coupled with memory. The one or more processors can activate the one or more actuators to cause the one or more actuators to increase a force applied to at least a portion of the lower limb to augment motion of the user.

### CROSS-REFERENCES TO RELATED APPLICATIONS

This application is a continuation and claims benefit and priority to International Patent Application No. PCT/US2024/044058, filed Aug. 27, 2024, which claims the benefit of priority under 35 U.S.C. § 119 to U.S. Provisional Patent Application No. 63/534,933, filed Aug. 28, 2023, each of which is hereby incorporated by reference herein in its entirety.

### BACKGROUND

Exoskeletons can be worn by a user to facilitate movement of limbs of the user. Exoskeletons can be custom fitted to the user.

### SUMMARY

This disclosure is generally directed to a custom exoskeleton configuration. For example, this technology can provide a soft ankle-foot exoskeleton. The soft ankle-foot exoskeleton can utilize a leg interface formed by a hybrid braided-composite shear architecture, with the sock of the user being a dynamic braided structure. In another example, this technology can provide an ankle-foot exoskeleton mechanically powered using a parallel-elastic actuator mechanism applied anteriorly upon the ankle-foot complex. The ankle-foot exoskeleton of the technical solution discussed herein can facilitate or provide the augmentation of movement, such as walking, running gaits, jumping, etc.

In one aspect, this disclosure is directed to a system for an ankle-foot exoskeleton of a user. The ankle-foot exoskeleton can include a braided structure configured to at least partially encapsulate a lower limb of the user, the braided structure configured to extend from at least below a knee of the lower limb of the user to at least above an ankle of the lower limb. The ankle-foot exoskeleton can include one or more actuators mechanically integrated with the braided structure. The ankle-foot exoskeleton can include one or more processors, coupled with memory. The one or more processors can activate the one or more actuators to cause the one or more actuators to increase a force applied to at least a portion of the lower limb to augment motion of the user.

The braided structure can comprise a composite shear interface formed of a plurality of materials. The braided structure can comprise a hybrid braided structure. The braided structure can comprise a hybrid braided-composite shear interface. The braided structure can be configured to fully encapsulate a circumference of the lower limb.

The ankle-foot exoskeleton can comprise a plurality of actuators mechanically integrated with the braided structure. The ankle-foot exoskeleton can comprise the one or more processors to cause the plurality of actuators to apply a compression force to the lower limb to augment the motion of the user. The plurality of actuators can be configured to monotonically increase a compression force as an actuation force increases to resist the actuation force without slippage via an applied shear force on the lower limb.

The ankle-foot exoskeleton can comprise the one or more actuators comprising a composite plate formed of a braided filament material and a bonding material configured to couple the braided filament material of the composite plate to the braided structure. The bonding material can comprise an epoxy resin. The one or more actuators can comprise the composite plate that fills in a resin to mount the one or more actuators to the braided structure. The braided structure can comprise a plurality of layers.

The ankle-foot exoskeleton can comprise the braided structure comprising a plurality of filaments, wherein at least one of the plurality of filaments comprises a sensor. The one or more processors can receive data from the sensor. The one or more processors can determine an amount of the force to apply based on the data. The one or more processors can activate the one or more actuators to apply the determined amount of force. The ankle-foot exoskeleton can comprise an inertial measurement unit configured to determine a shank angle between a shank of the braided structure and a foot of the lower limb. The ankle-foot exoskeleton can comprise the one or more processors to determine, based on the shank angle determined via the inertial measurement unit, an ankle angle during actuation of the one or more actuators.

In an aspect, this technical solution can be directed to a method for an ankle-foot exoskeleton of a user. The method can include providing a braided structure configured to at least partially encapsulate a lower limb of the user, the braided structure configured to extend from at least below a knee of the lower limb of the user to at least above an ankle of the lower limb. The method can include providing one or more actuators mechanically integrated with the braided structure. The method can include activating, by one or more processors coupled with memory, the one or more actuators to cause the one or more actuators to increase a force applied to at least a portion of the lower limb to augment motion of the user.

The braided structure can comprise a composite shear interface formed of a plurality of materials. The braided structure can comprise a hybrid braided structure. The braided structure can comprise a hybrid braided-composite shear interface. The braided structure can be configured to fully encapsulate a circumference of the lower limb.

The method can include a plurality of actuators mechanically integrated with the braided structure. The method can include causing, by the one or more processors, the plurality of actuators to apply a compression force to the lower limb to augment the motion of the user. The method can include monotonically increasing, by the plurality of actuators, a compression force as an actuation force increases to resist the actuation force without slippage via an applied shear force on the lower limb. The one or more actuators can comprise a composite plate formed of a braided filament material and a bonding material configured to couple the braided filament material of the composite plate to the braided structure.

The bonding material can comprise an epoxy resin. The one or more actuators can comprise the composite plate that fills in a resin to mount the one or more actuators to the braided structure. The braided structure can comprise a plurality of layers. The braided structure can comprise a plurality of filaments, wherein at least one of the plurality of filaments comprises a sensor. The method can include receiving, by the one or more processors, data from the sensor. The method can include determining, by the one or more processors, an amount of the force to apply based on the data. The method can include activating, by the one or more processors, the one or more actuators to apply the determined amount of force.

The method can include determining, by an inertial measurement unit, a shank angle between a shank of the braided structure and a foot of the lower limb. The method can include determining, by the one or more processors, based on the shank angle determined via the inertial measurement unit, an ankle angle during actuation of the one or more actuators.

In an aspect, this technical solution can be directed to a system for an ankle-foot exoskeleton of a user. The system can include a shin guard with a calf strap configured to couple to a lower limb of the user. The system can include a yoke structure at a lateral and medial side of a foot of the lower limb. The system can include a parallel-elastic actuator comprising a lateral beam and a medial beam coupled to the yoke structure. The system can include one or more processors, coupled with memory, to: actuate the parallel-elastic actuator into a state of dorsiflexion to augment motion of the user.

The parallel-elastic actuator can be configured with one degree of freedom. The parallel-elastic actuator can be configured with two degrees of freedom. The parallel-elastic actuator can comprise at least one of a bend or a coil.

### BRIEF DESCRIPTION OF THE DRAWINGS

The details of one or more implementations of the subject matter described in this specification are set forth in the accompanying drawings and the description below. Other features, aspects and advantages of the subject matter will become apparent from the description, the drawings and the claims.

FIG. 1 illustrates a schematic diagram of a lower limb exoskeleton.

FIG. 2 illustrates a powered ankle-foot exoskeleton comprising a 1-degree-of-freedom (DOF) uni-directional actuator and a hybrid braided-composite shear interface.

FIG. 3 illustrates a powered ankle-foot exoskeleton including a 1-DOF bi-directional actuator and a hybrid braided-composite shear interface.

FIG. 4 illustrates a plantar foot center-of-pressure trajectory.

FIG. 5 illustrates a powered ankle-foot exoskeleton including a 2-DOF uni-directional actuator and a hybrid braided-composite shear interface.

FIG. 6 illustrates a rendering of the powered ankle-foot exoskeleton including a 2-DOF uni-directional actuator and a hybrid braided-composite shear interface.

FIG. 7 illustrates a powered ankle-foot soft exoskeleton comprising a 2-DOF bi-directional actuator and a hybrid braided-composite shear interface.

FIG. 8 illustrates a 1-DOF bi-directional ankle-foot exoskeleton comprising a parallel-elastic actuator.

FIG. 9 illustrates 1-DOF bi-directional ankle-foot exoskeleton comprising a parallel-elastic ankle actuator with an unpowered subtalar inversion/eversion DOF.

FIG. 10 illustrates 1-DOF Bi-directional Ankle-foot Exoskeleton Comprising a Parallel-elastic Actuator in a state of Dorsiflexion.

FIG. 11 illustrates 1-DOF Bi-directional Ankle-foot Exoskeleton Comprising a Parallel-elastic Actuator in a state of Plantar Flexion.

FIG. 12 illustrates 2-DOF Bi-Directional Ankle-foot Exoskeleton Comprising Differential Parallel-elastic Actuation.

FIG. 13 illustrates The 2-DOF Bi-Directional Ankle-foot Exoskeleton Comprising Differential Parallel-elastic Actuation in a State of Eversion.

FIG. 14 illustrates The 2-DOF Bi-directional Ankle-foot Exoskeleton Comprising Differential Parallel-elastic Actuation in a State of Inversion.

FIG. 15 illustrates The 2-DOF Bi-directional Ankle-foot Exoskeleton Comprising Differential Parallel-elastic Actuation in a State of Dorsiflexion and Plantar Flexion.

FIG. 16 illustrates a flow diagram of an example method for augmenting motion of a user via an ankle-foot exoskeleton.

Like reference numbers and designations in the various drawings indicate like elements.

### DETAILED DESCRIPTION

This disclosure is generally directed to a custom exoskeleton configuration. For example, this technology can provide a soft ankle-foot exoskeleton. The soft ankle-foot exoskeleton can utilize a leg interface formed by a hybrid braided-composite shear architecture, with the sock of the user being a dynamic braided structure. In another example, this technology can provide an ankle-foot exoskeleton mechanically powered using a parallel-elastic actuator mechanism applied anteriorly upon the ankle-foot complex. The ankle-foot exoskeleton of the technical solution discussed herein can facilitate or provide the augmentation of movement, such as walking, running gaits, jumping, etc.

### I. Exoskeleton Overview

Exoskeletons (e.g., lower limb exoskeleton, knee exoskeleton, back exoskeleton, etc.) can include devices worn by a person to augment physical abilities. Exoskeletons can be considered passive (e.g., not requiring an energy source such as a battery) or active (e.g., requiring an energy source to power electronics and usually one or many actuators). Exoskeletons may be capable of providing large amounts of force, torque and/or power to the human body in order to assist with motion.

Exoskeletons can transfer energy to the human and may not interfere with the natural range of motion of the body. Exoskeletons can convert the energy source into useful mechanical force, torque or power. Onboard electronics (e.g., controllers) can control the exoskeleton. Output force and torque sensors can also be used to make controlling easier.

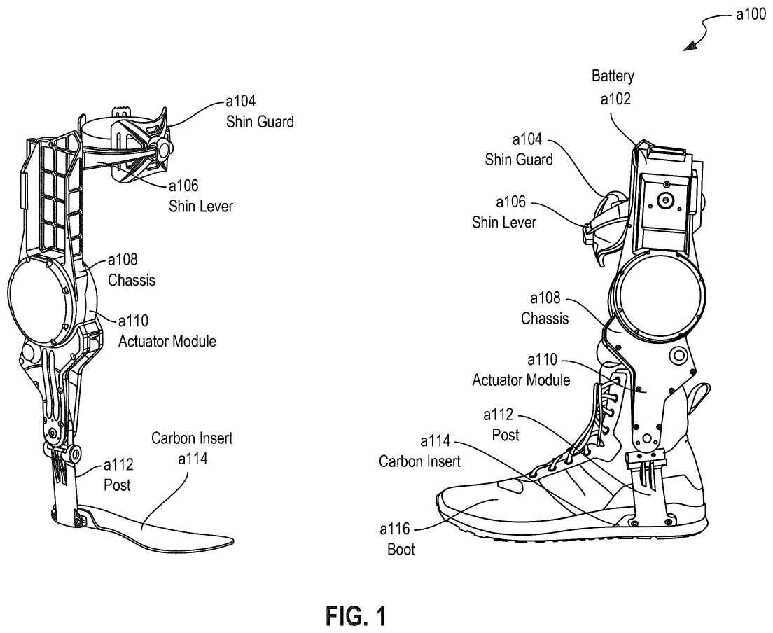

FIG. 1 illustrates a schematic diagram of a lower limb exoskeleton a100 (e.g., lower limb exoskeleton assembly, lower limb exoskeleton system, exoskeleton boot, mechanical exoskeleton, exoskeleton device, etc.). The exoskeleton a100 can include a battery a102 that can include an assembly that is installed onto the actuator module and supplies electrical energy to the system. The exoskeleton a100 can include a shin guard a104 that can include a part of the assembly that interfaces with the user's shin. The exoskeleton a100 can include a shin lever a 106 that can include a mechanical structure that connects the shin guard to a chassis. The chassis a108 can include a mechanical structure that connects the static components. The exoskeleton a100 can include an actuator module a110 that can include all the component in the lower limb exoskeleton assembly excluding the boot. The exoskeleton a100 can include a post a112 that can include a mechanical structure that connects to the boot. The exoskeleton a100 can include a carbon insert a114 that can include a carbon fiber structure located inside of the sole of the boot. The exoskeleton a100 can include a boot a116 that connects to the user and the actuator module. The exoskeleton a100 can include a spool shaft that can include a shaft that is driven by the motor and winds the belt around itself. The belt can include a tensile member that is pulled by the spool shaft and applies a force to the ankle lever. The exoskeleton a100 can include an ankle lever that can include a lever used to transmit torque to the ankle. Lower limb exoskeletons can be used to augment the ankle joint.

The lower limb exoskeleton a100 can include a rugged system used for field testing. The lower limb exoskeleton a100 can include an integrated ankle lever guard (e.g., nested lever). The lower limb exoskeleton a100 can include a mechanical shield to guard the belt and ankle lever transmission from the environment. The housing structure of the system can extend to outline the range of travel of the ankle lever on the lateral and medial side. The lower limb exoskeleton a100 can include a shin lever self-centering mechanism. A self-centering mechanism can be incorporated into the shin lever. Degrees of freedom can be incorporated into the lower limb exoskeleton to reduce skin sheer and increase the comfort to the user. The lower limb exoskeleton a100 can include a self-centering mechanism to push the shin lever to the shin lever's center of travel if the shin lever is not already there. This mechanism can be composed of one or more springs. The self-centering mechanism on the lower limb exoskeleton can use repelling magnets to push the shin lever to its center of travel. The magnets can be attracted each other and pull the shin lever to its center of travel.

### II. Powered Exoskeletons Comprising a Hybrid Braided-Composite Shear Interface

According to the systems and methods of the present disclosure, an exoskeleton can augment a user's activities. Rigid and compliant structures can be integrated directly into footwear. This can allow for benefits for the purposes of human augmentation. For example, these structures can include a way to apply force to the ankle and lower limbs without injury or discomfort.

In some embodiments, the systems and methods can include or provide a soft ankle-foot exoskeleton. One or more exoskeletal actuators (e.g., sometimes referred to generally as actuators) of the exoskeleton can apply forces on the lower leg in shear, where the applied body force can be tangential to the surface of the body of the user. In such cases, four ankle-foot exoskeleton configurations can be provided. Each of the configurations can involve the actuator forces applied to the leg in shear, covering 1-degree-of-freedom DOF or 2-DOF, and uni-directional or bi-directional actuator mechanisms. The 1-DOF configuration can actuate the ankle joint unidirectionally or bi-directionally. The 2-DOF configuration can actuate the ankle unidirectionally or the subtalar joint bi-directionally, and both the ankle and subtalar joints bi-directionally. For each configuration, actuation can span from a leg composite actuator plate to a plantar foot plate. Each composite actuator plate can be integral with a braided sock formed around the biological leg, where each plate can be a composite including the braided filament material held together by an epoxy resin, among other similar bonding materials. Beyond the actuator plate or plates, the sock can include or correspond to a dynamic braided structure. In such cases, the leg interface can be formed by a hybrid braided-composite shear architecture, where the number of composite actuator plates can be equal to the number of actuators. When each actuator applies a force via each actuator composite mounting plate, the braided sock can proportionally increase the compressive load of the sock around the biological leg, with compressive force monotonically increasing with increasing actuator force, to effectively resist the actuator force without slippage through an applied shear force on the body. When the actuator or actuators no longer apply force, the compressive force on the leg can be minimized, for instance, to increase comfort. Uni-directional actuators can apply torque to enhance late stance ankle and subtalar dynamics in gait. Bi-directional actuation can allow (or enable) ankle-foot augmentation throughout all phases of gait. In various embodiments, the hybrid braided-composite architecture can include leg sensing modalities for the estimation of foot-ankle-leg kinematics, kinetics, muscle-tendon dynamics, or activations. Additional sensors can estimate exoskeleton positions, speeds, forces, or temperatures. The sensory data can inform microprocessor feedback control commands to each actuator of the soft ankle-foot exoskeleton for the augmentation of bipedal motor tasks (e.g., movements), such as walking, running, or jumping, among others.

In some embodiments, the systems and methods can include an ankle-foot exoskeleton that is mechanically powered using a parallel-elastic actuator mechanism applied anteriorly upon the ankle-foot complex, such as for the augmentation of walking, running gaits, jumping, or other tasks. Two ankle-foot exoskeleton configurations can be provided, covering 1-DOF and 2-DOF actuator configurations, each with bi-directional or parallel-elastic actuation. The 1-DOF embodiment can actuate the ankle joint bi-directionally. The 2-DOF can actuate both the ankle and subtalar joints bi-directionally. For the 1-DOF configuration, elastic strain energy stored in mediolateral bending beams can be modulated by a parallel-motor system to enhance late stance-powered plantar flexion about the ankle joint. For the 2-DOF configuration, strain energies stored in medial and lateral bending beams can be modulated independently by medial and lateral parallel-motor systems, respectively, throughout the gait cycle to enhance ankle and subtalar dynamics throughout the late stance phase of walking, running, jumping gaits, or other movements. Each ankle-foot exoskeleton configuration with parallel-elastic actuation can include leg sensing modalities for the estimation of foot-ankle-leg kinematics, kinetics, muscle-tendon dynamics, or activations. Additional sensors can estimate exoskeleton positions, velocities, forces, temperatures, or other information. These sensory data can inform microprocessor feedback control commands to each parallel-elastic actuator for the augmentation of bipedal motor tasks such as walking, running, or jumping, among others.

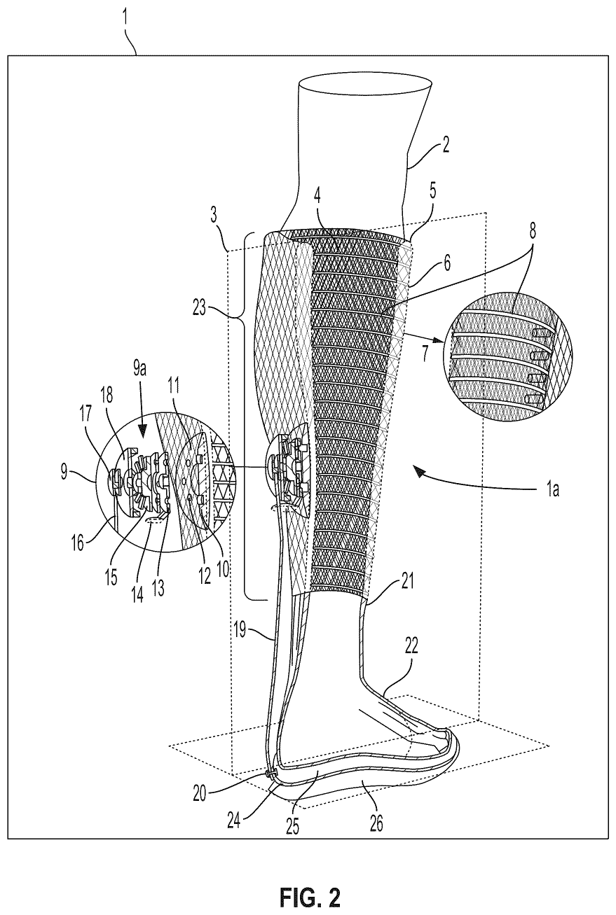

FIG. 2 shows a soft ankle-foot powered exoskeleton including a hybrid braided-composite shear interface and a unidirectional actuator for the control of ankle plantar flexion during the late stance phase. For this configuration, a braided structure (e.g., braided structure (1a)) can be formed around the contours of the biological leg, extending from above the calf muscle bulge at its upper proximal aspect, to above (e.g., immediately above) the ankle joint at its lower or distal aspect. The braided structure (1a) can at least partially encapsulate a lower limb of the user. The lower limb can refer to a part of the body at or below at least the hip of the user, for example. In some cases, the braided structure (1a) may be formed around the lower limb of the user, conforming to the shape or structure of the lower limb, including any additional attachment or wearable component equipped by the user, for example. For instance, the braided structure (1a) may be formed around clothing worn by the user, leg wrap, or other equipment, accessories, or devices thereof. The braided structure (1a) can be formed with nylon, polyester filaments, or other types of materials (e.g., fiber materials). The braided structure (1a) may be formed around other parts (e.g., additional or alternative parts) of the lower limb, extending from at least below the knee of the lower limb to at least above the ankle of the lower limb. The braided structure (1a) may fully encapsulate the circumference of the lower limb (or the circumference of a portion of the lower limb). In some cases, the braided structure (1a) may extend above the knee or below the ankle of the lower limb. To optimize the leg gripping force for a given actuator loading, the fiber orientation can include or correspond to +/−45 degrees, +/−60 degrees, or other fiber orientations. The braided structure can be digitally fabricated, such as from pre-determined leg molds or a digital scan of the leg anatomical contours of the user. For such a braided weave fabrication, digitally controlled braiding machines can be utilized for the fabrication.

FIG. 2 can include a unidirectional plantar flexion ankle exoskeleton (1) with a braided weave structure (1a) and composite actuator mounting plate (10) that surrounds a human lower leg (2). The unidirectional plantar flexion ankle exoskeleton may sometimes be referred to as an exoskeleton (1) including the braided weave structure (1a) and the composite actuator mounting plate (10). The braided weave structure (1a) may sometimes be referred to as a braided structure 1a. The composite actuator mounting plate (10) may sometimes be referred to as a composite actuator plate (10), actuator mounting plate (10), or a mounting plate (10). The mounting plate (10) can be positioned, located, or configured posterior to the braided structure (1a) (e.g., at the back or rear of the lower limb of the user). In some arrangements, the exoskeleton (1) can include one or more mounting plates coupled to other parts of the braided structure (1a), e.g., posteriorly, anteriorly, or medially.

A cross-sectional view along the sagittal plane (3) can be shown in, but not limited to FIG. 2, with an interior layer (4) of the braided structure (1a) (e.g., woven braided sleeve structure), double-backed proximal aspect (5) of the braided structure (1a), and an exterior layer (6) of the braided structure (1a) (e.g., braided sleeve structure). An inverted upper braided sleeve (7) can be shown. An elastomeric skin-interfacing material extruded onto the surface of the interior face of the braided structure (1a) can be used to increase the coefficient of friction between the interior layer of the braided structure (1a) and the skin of the user for maximal gripping during shear actuation (8), for example.

A view (9) of an actuator assembly (9a) and composite actuator plate (10) can be shown in FIG. 2. The actuator assembly (9a) can correspond to an actuator (9a) capable of or configured to mount to the composite actuator plate (10) (e.g., sometimes referred to as a composite plate), for example. An epoxy resin cast (e.g., bonding material) within the braided sleeve (e.g., braided structure (1a)) can form a composite actuator plate (10). The cast resin can cure and mechanically lock securely around the braided filaments (11) to form the composite actuator plate (10). The braided filaments (11) can refer to braided filament material formed by interweaving multiple filaments (or fibers) in a braided pattern, for example. The braided filament material can be composed of one or multiple materials, such as at least one of but not limited to carbon fiber, glass fiber, aramid fiber, polyester, or other types of materials discussed herein. In some cases, the actuator (9a) (or other actuators) can include the composite actuator plate (10) that fills in a resin to mount the actuator (9a) to the braided structure (1a). The actuator (9a) can be mounted in response to the cast resin curing and mechanically locking the actuator (9a) to a portion of the braided structure (1a), for example.

The composite actuator plate (10) may be formed using other materials (e.g., other bonding materials), not limited to epoxy resin, and using at least one suitable casting or formation technique, such as injection molding or casting, 3D printing, compression molding, etc. The bonding material can couple the braided filament material (e.g., braided filament (11)) of the composite actuator plate (10) to the braided structure (1a). By forming the composite actuator plate (10) within the braided structure (1a), the actuator plate (10) can be mechanically integrated with the braided structure (1a). By coupling the actuator (9a) to the actuator plate (10) or forming the actuator (9a) in connection with the actuator plate (10), the actuator (9a) can be mechanically integrated with the braided structure (1a). Threaded inserts cast into the composite may be used to secure the actuator assembly (9a) onto the braided sleeve (12).

The actuator assembly (9a) can include at least one of but not limited to an actuator mounting base (13), cord (e.g., tensile element) winder guide (14), stator and printed circuit board (PCB) (e.g., outrunner style brushless DC motor) (15), cord (e.g., tensile element) (16), winch pulley (17), rotor (e.g., outrunner style brushless DC motor) (18), or cord (e.g., tensile element) transitioning into a synthetic tendon (19). The synthetic tendon can be mechanically attached to a composite underfoot (plantar) plate (20) at its posterior aspect. Under computer control (e.g., the control system), the motor of the actuator (9a) (or multiple actuators) may be controlled to modulate (e.g., increase or decrease) the tensile force borne by the cord and synthetic tendon (19) or the force applied to the posterior aspect of the plantar foot plate (20), for instance, to modulate the plantar flexion torque about the biological ankle joint during walking, running, jumping gaits, among others. The force generated by the motor of the actuator (9a) can be applied to other parts of the lower limb of the user based on at least one of the location of the actuator (9a) or the connection of the cord and synthetic tendon (19) relative to the plantar foot plate (20), such as at the posterior, anterior, or medial aspect of the footplate.

Although examples herein provide that the motor of the actuator (9a) applies force to the footplate via the cord and synthetic tendon (19), it should be noted that the motor can apply force to other components or structure of the exoskeleton (1) connected to the actuator (9a) via the cord and synthetic tendon (19), for example. Additionally or alternatively, under the computer control (e.g., the control system), the motor of the actuator (9a) (or multiple actuators) can be controlled to modulate (e.g., increase or decrease) a compression force borne by the cord and synthetic tendon (19) or the compression force applied to the plantar foot plate (20) or the lower limb to augment the motion of the user.

The computer system (e.g., the control system) can control multiple motors of a plurality of actuators to apply a force (e.g., compression or tensile force) to the plantar foot plate (20) or other portions of the lower limb to augment motion of the user. In some cases, when each actuator (9a) applies a force via a corresponding actuator composite mounting plate, the braided structure (1a) can proportionally increase the compressive/compression load/force around the lower limb of the user. The control system may command the actuators (9a) to monotonically increase the compression force around the lower limb of the user as an actuation force increases (e.g., actuation event), e.g., to effectively resist the actuator force without slippage via an applied shear force (e.g., shear gripping force discussed herein) on the lower limb of the user. The shear force can refer to a force that acts parallel to the cross-section of a material, causing one part of the material to slide past another. In this case, the shear force can be utilized to assist with gripping and avoid slippage, for instance, by monotonically increasing the compression force around the lower limb to resist the applied (e.g., sensed or measured) shear force. The actuation force may be relative to the ankle or other joints of the lower limb. The increase (or decrease) of the actuation force can be measured by at least one force sensor. The control system can obtain the measured actuation force from the sensor to command the one or more actuators (9a) to increase (or decrease) the compression force (or other types of force) according to the detected actuation force.

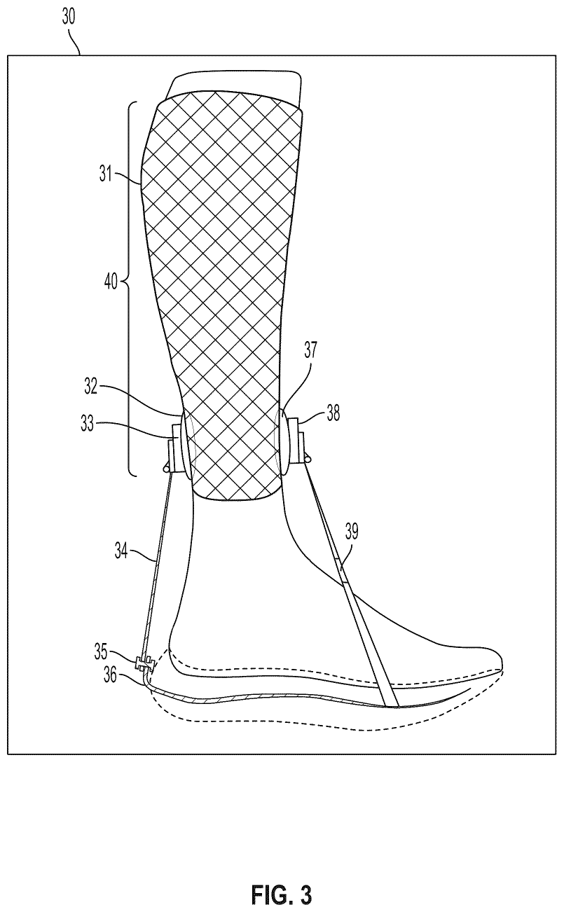

The actuator (9a) can be mounted or coupled to the actuator plate (10) (e.g., mounting plate). The actuator plate (10) may support mounting one or more actuators (9a). The actuator (9a) can be decoupled or dismounted from the actuator plate (10). In some configurations, the actuator plate (10) may be repositioned to other parts of the braided structure (1a), e.g., while disconnected from the actuator (9a). In some other configurations, the actuator plate (10) can be in a fixed location or position within the braided structure (1a) after the formation or casting. In certain configurations, the actuator (9a) may be locked or fixed to the actuator plate (10), e.g., the actuator (9a) may be assembled or formed along with the actuator plate (10). Although FIG. 2 shows one actuator (9a), it should be noted that the exoskeleton (1) or the braided structure (1a) can include or be coupled to multiple actuators (9a), such as described in conjunction with at least one of FIG. 3, 5-7, etc.

The exoskeleton (1) can include a system, a device, or a processing unit (not shown) comprising one or more processors, coupled with memory. The system may correspond to or be referred to as a control system, a microprocessor, a chip, a data processing system, or other systems composed of hardware or a combination of hardware and software components. For purposes of providing examples, the system comprising the one or more processors can be referred to as a control system (not shown) herein.

The control system can include an interface configured to communicate with one or more components of the exoskeleton (1), including at least one of one or more sensors or one or more components of the actuator (9a), among others. The interface of the control system can include wired or wireless connections. Connections can be established using a variety of communication protocols (e.g., TCP/IP, Ethernet, ARCNET, SONET, SDH, Fiber Distributed Data Interface (FDDI), IEEE 802.11a/b/g/n/ac CDMA, GSM, WiMax and direct asynchronous connections).

For example, the control system can receive sensor data (e.g., measurements) from the one or more sensors of the exoskeleton (1). The control system can provide instructions or signals to control the actuator (9a), e.g., command/instruct activation or operation of the actuator (9a), including the non-limiting exemplary operations as described herein. The control system can process information (e.g., sensor data) to determine whether to operate the actuator (9a) or the types of operations to initiate for the actuator (9a). The one or more operations can include but is not limited to activating or deactivating the actuator (9a), increasing or decreasing the force from the actuators (9a) applied to at least a portion of the lower limb (to augment motion of the user), etc. Activating the actuator (9a) can include or involve causing the actuator (9a) to increase a force applied to at least a portion of the lower limb of the user, equips with or couples to the exoskeleton (1), to augment user motion. Deactivating the actuator (9a) can involve causing the actuator (9a) to decrease the force applied to at least the portion of the lower limb to reduce motion augmentation or terminate augmenting the user motion, for example. In some cases, the control system can process the sensor data to determine the amount of force to apply for augmenting motion for the user. The control system can activate the actuator (9a) to apply the determined amount of force or a predetermined amount of force, for example. The control system can command the actuator (9a) to perform other operations discussed herein.

The control system (not shown) can be embedded into the exoskeleton (1). For example, the control system can be coupled to or be a part of the actuator assembly (9a). In another example, the control system can be coupled to (or formed within) at least a portion of the braided structure (1a) and interfaces with the actuator (9a). In certain configurations, the control system may be remote from the exoskeleton (1), such as a separate component or device from the exoskeleton (1). The actuator (9a) may be connected to the control system via a wired or wireless connection. For instance, the control system may be a client device (e.g., phone, tablet, or laptop) operated by the user to control at least the actuator (9a). In this example, the control system can provide the user with a user interface (e.g., graphical user interface (GUI) or analog interface) to control the operation of the actuator (9a). The user interface can include at least one of interactive element, a graphical display, analog buttons, etc. The control system may allow the user to control the operation of the actuator (9a) manually, e.g., activate or deactivate the actuator (9a) according to the user selection of the operation. The control system may request authorization from the user to perform automatic operations, such as operating the actuator (9a) according to the sensor data, where the operation can be determined by the control system based on processing the sensor data.

FIG. 2 can include or show a transitional area (21) where the exterior and interior bottom edges of the braided sleeve are joined to a knitted sock structure (22). The braided structure (1a) (or the braided sleeve) can include a plurality of layers. For example, the braided structure (a) can include or be formed by interior (4) and exterior (6) layers of woven braided structure or component combined with the composite actuator plate (10) can include or form a hybrid braided-composite shear interface (23). For instance, the hybrid braided-composite shear interface (23) may correspond to a composite shear interface. The braided structure (1a) can include the composite shear interface formed of multiple materials (e.g., different layers, components, or materials joined together, across which shear forces are produced or transmitted). In another example, the hybrid braided-composite shear interface (23) may correspond to a hybrid braided structure. In this example, the braided structure (1a) may include the hybrid braided structure formed of one or more types of fibers or various other materials that are interweaved (or braided).

The proximal aspect of the hybrid braided-composite shear interface (23) (e.g., composite shear interface or hybrid braided structure) can be positioned above the calf muscle bulge for a robust shear gripping force, among other locations or positions of the lower limb, for example. The hybrid braided-composite shear interface (23) can be composed or formed of one or more materials, including but not limited to at least one of adhesive material, resin, metal, fabric, etc. The hybrid braided-composite shear interface (23) can be formed or composed of a combination or different combinations of materials. The composite plantar foot plate (24) can be sandwiched between an upper cushioning material (above the composite plate) (25), and a lower cushioning material (below the composite plate) (26).

The embedded control system and PCB (15) may undergo a closed-loop control, where a microprocessor receives sensory information from wearable sensors to modulate, in an updating manner, the ankle plantar flexion torque. The wearable sensors can include, but are not limited to, at least one of the following sensing modalities: joint position sensors; limb translational position sensors, motor position sensors; tensile mechatronic force sensors; plantar-foot pressure sensors; muscle activation sensors; muscle length, velocity, and force sensors; muscle stiffness sensors; or skin strain sensors.

Inertial measurement units (IMUs) can be used for the measurement of ankle-foot and leg kinematics, such as ankle, subtalar, and knee joint positions; shank and foot roll, pitch and yaw angular positions; global frame ankle and knee translational positions; or the derivatives of these angular and translational kinematic positions, to name a few. For example, an IMU can be used to measure a shank angle between a shank (e.g., from the knee to the ankle) of the braided structure (1a) and a foot of the lower limb. The shank angle can refer to the angle of the shank relative to a vertical in the sagittal plane, for example. In some configurations, the IMU can provide the measured shank angle to the control system. The control system can determine an ankle angle based on the shank angle determined via the IMU. The control system can determine the ankle angle during the activation/actuation of the one or more actuators (9a) to control the operation of the one or more actuators (9a). For example, the ankle angle of the user during a motion (e.g., walking, running, or jumping) can be indicative of the positioning of the lower limb during the motion. Depending on at least one of the ankle angle, changes between a plantarflexion and a dorsiflexion, shank angle, movement of the user (e.g., forward, backward, or side-to-side), etc., the control system may activate at least one actuator (9a) to increase or decrease the compression force applied to at least a portion of the lower limb to facilitate the intended or desired action of the user, including but not limited to lifting or lowering the toe or heel of the user, for example. For instance, when the ankle is in dorsiflexion (e.g., toes moving upwards), the control system can activate at least one actuator (9a) to apply a controlled force assisting the dorsiflexion movement. In another example, as the user transitions from heel strike to toe-off, the control system may command the at least one actuator (9a) to adjust the stiffness of the ankle joint, such as depending on the measured angle. Adjusting the stiffness can provide the user with a relatively more natural walking experience. In further examples, if the ankle angle indicates the start of the push-off phase (e.g., plantarflexion), the control system can activate the at least one actuator (9a) to apply a force to assist in propelling the user (e.g., the lower limb of the user) forward. The control system can manage other operations of at least the actuators (9a) of the exoskeleton (1) according to the IMU data. One or more encoders can be used to measure motor positions or velocities. Other sensors may be used to determine the position, velocity, or torque of the motor. It should be noted that the ankle angle can be used in conjunction with other types of sensor data, including but not limited to the acceleration, direction, or velocity data indicating the movement of at least a portion of the lower limb of the user, to assist or enhance the user movement.

The wearable sensors may include force and pressure sensors. A force sensor can be placed in series with the tensile cord and synthetic tendon (19), for instance, to close the loop on cord or tendon force, or the applied ankle torque by the exoskeleton. In some cases, an insole pressure sensing array can be used to estimate at least one of but not limited to: the vertical ground reaction force, or the foot-exoskeleton center of pressure trajectory. Such an insole sensing array can be integral to the knitted (sock) structure (22) or the hybrid braided-composite interface (23) that forms the sock, for instance, in FIG. 2. These signals can be used in combination with IMUs to estimate or determine the biological ankle torque using an inverse dynamics calculation. By sensing vertical force and center of pressure position, a machine learning method can be used or implemented to estimate the horizontal components of the ground reaction force vector in real time (e.g., during utilization of the exoskeleton). Such a machine learning method can include the use of biomechanical gait data to train a neural net function. With knowledge of ankle position, the center of pressure position and the ground reaction force vector, the perpendicular moment arm from the ankle rotational axis to the line of force of the ground reaction force can be estimated or determined. The biological ankle torque can be computed from the magnitude of the ground reaction force multiplied by the ankle moment arm. The desired exoskeleton torque can be functionally related to the biological ankle torque in a power steering framework, where the user can modulate exoskeletal assistance by modulating the biological ankle torque. In some cases, the center of pressure position versus time can be employed to determine an optimal ankle torque trajectory applied by the exoskeleton using an apriori relationship between center-of-pressure trajectory signal features and optimal exoskeletal torque output.

The wearable sensors may include at least one of muscle electromyography (EMG) sensors, accelerometer sensors, ultrasound sensors, magnetometer sensors, acoustic sensors, muscle stiffness sensors, strain sensors, or other sensors to estimate muscle-tendon lengths, speeds, or forces. These sensing modalities can be integral to the knitted structure (22) or the hybrid braided-composite interface (23) that forms the sock, such as in FIG. 2. The filament fibers that are digitally braided can have sensing capabilities, or sensors can be attached to the braided structure (1a) in muscle regions to serve as inputs for the control of the uni-directional plantar flexion actuator. For example, the braided structure (1a) can include a plurality of filaments (e.g., fiber strands). At least one or a portion of the filaments can include or be coupled to at least one sensor, for instance, to measure or monitor the muscle-tendon or other activities of the user. The filaments of the braided structure (1a) may house or include other sensors to monitor the muscle activities of the user, for example. Data from the one or more sensors can be transmitted or provided to the control system. In some implementations, microphone fibers may be used to noninvasively track muscle-tendon acoustic frequency response. This can be performed, for example, by measuring the natural acoustic properties of the tendon and muscle when these organs are dynamically changing or during activation. The resulting acoustic behavior can be used to estimate muscle activity or force output. Such a microphone fiber can be used as the filament of the braided structure (1a), such as in FIG. 2. In some cases, in some embodiments, the braided filaments can allow (or enable) skin strain sensing for measurement of muscle shape and contractile activation. Additionally or alternatively, the braided structure 1a can house EMG electrodes to estimate muscle activation of underlying muscle. Through an estimation of muscle length and speed from wearable IMUs positioned distal and proximal to the ankle and knee joints, the calf muscle length and speed, for example, can be estimated. Using a biophysical muscle model, calf muscle state combined with muscle activation can be used to estimate muscle force. In some cases, in a tendon-tapping approach or configuration, an IMU can be used to measure Achilles tendon velocity wave speed which correlates to calf muscle-tendon force. In this case, an actuator, such as a vibratory motor, can apply disturbances to the tendon, and an IMU can be used to measure the tendon wave speed response. Subsequently, the wave speed can be calibrated to muscle-tendon force. In some cases, noninvasive ultrasound sensors integrated into the woven braid can be used to estimate muscle length and/or speed. The information regarding the muscle state, combined with muscle activation, can be used to estimate muscle-tendon force using a biophysical muscle model.

In some configurations, the control system can receive data from the one or more sensors attached to the filaments of the braided structure (1a), for example. The sensor data can include any muscle-tendon force (e.g., contraction, tensile force, or shear force) or motion/movement by the user. For example, the control system can process the muscle-tendon force sensed by at least one sensor indicative of the movement of the user and the magnitude of the force (e.g., exertion level, pressure, strain, or intensity) exerted to perform the movement. Based on at least the muscle-tendon force applied by the user (and sensed by at least one sensor), the control system can determine the amount of force to apply to the lower limb, e.g., to the composite plantar foot plate (24). The applied force can assist the user in augmenting motion. In response to the determination, the control system can activate one or more actuators (9a) to apply the determined amount of force, thereby augmenting motion for the user.

For the various configurations, approaches, or methods used to estimate muscle-tendon force, muscle-tendon force multiplied by joint moment arm can provide an estimate of biological joint torque. Using a power-steering control method, the desired exoskeleton torque can be functionally related to the biological joint torque, and a robotic closed-loop torque control can be applied to a servo to that desired torque. In various cases, deviations or changes can be made to the configuration of FIG. 2. For example, the braided-composite structure (1a) can continue throughout the ankle-foot complex to form a fully braided-composite sock. In another example, such as described in conjunction with but not limited to FIGS. 3-5, multiple actuators can be employed to exert torques about the ankle-foot complex to achieve ankle and subtalar uni and bidirectional actuation methods.

FIG. 3 illustrates a 1-DOF bi-directional soft ankle-foot exoskeleton (30) (e.g., sometimes referred to as exoskeleton (30)), such as similar to the soft exoskeleton configuration of at least FIG. 2, with an addition of a second actuator that dorsiflexes the ankle. In various embodiments, a powered ankle-foot exoskeleton (30) can include a 1-DOF bi-directional actuator (38) and a hybrid braided-composite shear interface (40), for example. Similar to the FIG. 2 configuration, for example, a braided sleeve structure (e.g., braided structure) (31) can be employed, e.g., sometimes referred to as braided sleeve structure (31) or braided structure (31). The braided structure (31) can include one or more features or functionalities similar to the braided structure (1a), described in conjunction with at least FIG. 2, for example. The posterior composite motor mounting plate (32) can be a connecting means of posterior actuator (33) to the braided structure (31) (e.g., braided shear structure (31)). The posterior tensile element (34) can extend from the posterior actuator (33) to the posterior tensile element mounting point (35) that connects the posterior tensile element (34) to the plantar composite footplate (36). Anteriorly, the anterior actuator composite mounting plate (37) can connect the anterior actuator (38) to the braided shear structure (31). The anterior tensile element (39) can span from the anterior actuator (38) to the plantar composite footplate (36). The braided sleeve (31) combined with the posterior (32) and anterior (37) composite actuator plates can form a hybrid braided-composite shear interface (40), where its proximal aspect may be positioned above the calf muscle bulge for a robust shear gripping force. In some cases, the sensing and control methods of FIG. 2 configuration may be applied to the soft-powered ankle-foot exoskeleton of FIG. 3 including a 1-DOF bidirectional actuator framework.

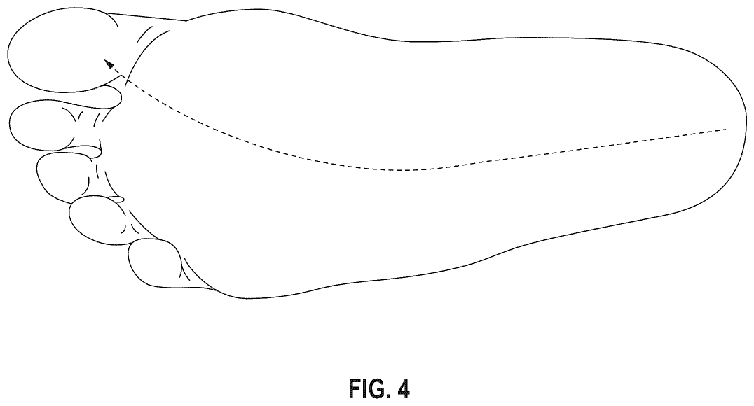

In some embodiments, the systems and methods can include a powered ankle-foot exoskeleton including a 2-DOF uni-directional actuator and a hybrid braided-composite shear interface. For example, the human ankle-foot complex can be approximated as a 2-DOF joint that rotates about 2 axes (or other axes), such as the ankle axis and the subtalar axis. These two axes can allow the foot to plantarflex or dorsiflex and invert or evert. During gait, the foot can rotate about both of these axes. In some cases, as the user progresses through the stance phase of gait, the center of pressure of the foot progresses in a non-linear fashion, such as in FIG. 4. FIG. 4 illustrates the plantar foot centre-of-pressure trajectory. The following ankle-foot exoskeleton configurations can utilize 2 actuated DOFs to apply torque to the ankle and subtalar axes to control the 2-DOF center of pressure trajectory of the foot and to allow (or enable) the augmented human biomechanics to more closely match the natural biomechanics.

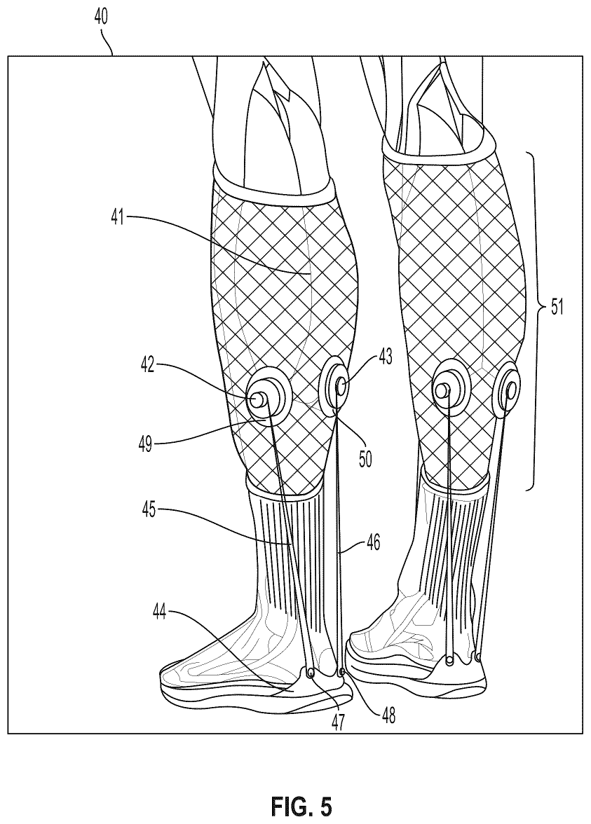

FIG. 5 illustrates a 2-DOF uni-directional soft ankle-foot exoskeleton (40) similar to the earlier soft exoskeletal configuration of at least FIG. 2, with two actuators attached posteriorly to the hybrid composite-shear structure (41) for the differential actuation of the ankle and subtalar joints of the ankle-foot complex. The hybrid composite-shear structure (41) may be referred to as a braided sleeve structure, braided shear structure, or braided structure. The braided sleeve structure (41) can be positioned above the calf muscle bulge for a robust shear interface. The lateral posteriorly-attached actuator (42) and medial posteriorly-attached actuator (43) can be attached to the plantar composite footplate (44) via lateral tensile element (45), or medial tensile element (46) via lateral posterior synthetic tendon swivel mounting point (47), or medial posterior synthetic tendon swivel mounting point (48). The lateral posteriorly-attached actuator (42) can be attached to the braided shear structure (41) via lateral composite actuator plate (49). The medial posteriorly-attached actuator (43) can be attached to the hybrid composite-shear structure (41) via medial composite actuator plate (50). Actuator composite plates (49) and (50) integral with the braided sleeve structure (41) can form a hybrid braided-composite shear interface (51). In some cases, at least one of the actuator composite plates (49) or (50) can be a part of the braided sleeve structure (41). In some other cases, at least one of the actuator composite plates (49) or (50) may be a component separate from the braided sleeve structure (41), e.g., the actuator composite plate (49) or (50) may be attached or coupled to the braided sleeve structure (41) via at least one suitable coupling mechanism, such as mechanical fastening, embedding, (e.g., adhesive) bonding, or other joint techniques.

In such configuration, a torque in the plantar flexion direction can be applied to the foot when both tensile actuators apply a force to the foot. When force is applied through one actuator, an eversion (lateral actuator), or inversion (medial actuator) torque can be applied in addition to the plantar flexion torque.

The unidirectional nature of this configuration can allow zero torque to be applied to the ankle and subtalar joints when there is no tension in the tensile elements (45) or (46). This can allow zero torque control when the system is powered off, for example. Because torque can be applied in both the plantar flexion, eversion, or inversion directions, torque can be modulated as the user progresses through the stance phase of a walking or running gait cycle. This can have the effect similar to changing the center of pressure between the user's shoe and the ground surface.

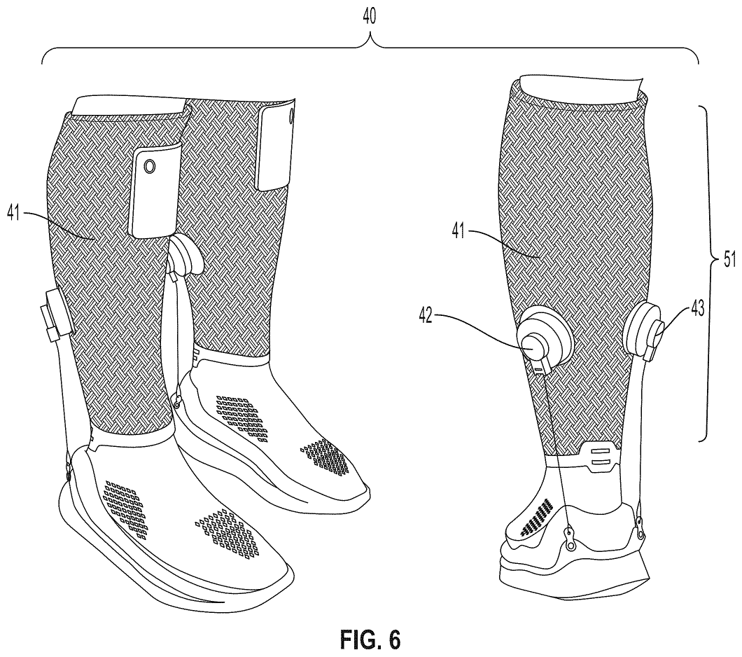

FIG. 6 illustrates the 2-DOF uni-directional soft ankle-foot exoskeleton (40) of FIG. 5 using a rendering. For example, FIG. 6 illustrates a rendering of the powered ankle-foot exoskeleton (40) including a 2-DOF uni-directional actuator (e.g., (42) or (43)) and a hybrid braided-composite shear interface (51), where the left rendering can include a battery pack assembly attached to the anterior aspect of the braided structure via a composite battery plate. The soft exoskeleton (40) of FIG. 6 can show at least one of a modular battery pack, a battery female receptacle, and a composite battery plate attached anteriorly to the braided structure. Similar to the composite actuator plates, the battery assembly can be mounted to the braided structure via an epoxy resin cast within the braided sleeve, forming a composite battery plate attachment means. The cast resin can cure and mechanically lock securely around the braided filaments. Similar to composite actuator plates, threaded inserts cast into the composite battery plate can be used to secure a battery female receptacle that receives the modular battery pack onto the braided sleeve. Wires can pass from the battery pack assembly, located on the anterior aspect of the soft exoskeleton, to the actuator assemblies passing between the braided interior and exterior layers. These interior and exterior braided layers may be depicted in but not limited to FIG. 2, for example, showing an interior layer (4) of woven braided sleeve structure (1a), double-backed proximal aspect (5) of the braided structure (1a), and an exterior layer (6) of the braided sleeve structure (1a).

The sensing and control methods outlined in configuration with at least FIG. 2 configuration can be applied to the soft-powered ankle-foot exoskeleton, such as shown in at least one of but not limited to FIG. 5 or FIG. 6, including the 2-DOF uni-directional actuator framework.

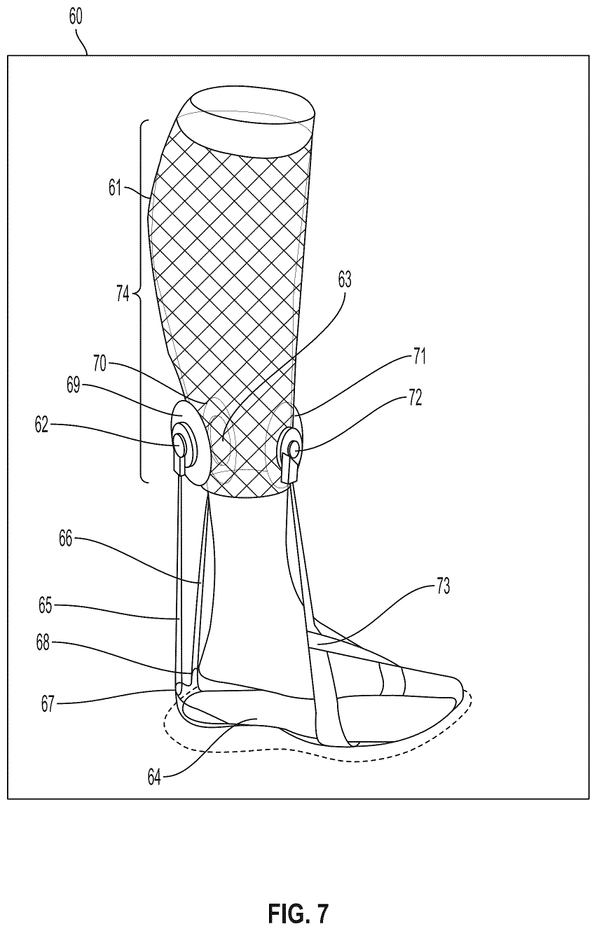

In various embodiments, the systems and methods can include a powered ankle-foot exoskeleton, including a 2-DOF bi-directional actuator and a hybrid braided-composite shear interface. FIG. 7 illustrates a 2-DOF bi-directional soft ankle-foot exoskeleton (60) similar to the soft exoskeletal embodiment of at least one of but not limited to FIG. 5 or 6, with an added actuator attached anteriorly to the braided sleeve structure (61) to create a bi-directional and differential actuation of the ankle and subtalar joints of the ankle-foot complex. Similar to other configurations, the braided sleeve structure (61) can extend significantly above the calf muscle bulge for a robust shear interface. The lateral posteriorly-attached actuator (62) and medial posteriorly-attached actuator (63) can be attached to the plantar composite footplate (64) via lateral tensile element (65) and medial tensile element (66) via lateral posterior synthetic tendon swivel mounting point (67) and medial posterior synthetic tendon swivel mounting point (68). The lateral posteriorly-attached actuator (62) can be attached to the hybrid composite-shear structure (61) via lateral composite motor plate (69). The medial posteriorly-attached actuator (63) can be attached to the hybrid composite-shear structure (61) via medial composite motor plate (70). Anteriorly, the anterior actuator composite mounting plate (71) can connect the anterior actuator (72) to the hybrid composite-braided structure (61). The anterior tensile element (73) can span from the anterior actuator (72) to the plantar composite footplate (64). Motor composite plates (69) and (70) integral with the braided sleeve structure (61) can form a hybrid braided-composite shear interface (74).

The sensing and control methods, such as those described in conjunction with at least FIG. 2 configuration, can be applied for the soft powered ankle-foot exoskeleton of FIG. 7 including a 2-DOF bi-directional actuator framework. The battery pack assembly, such as shown in at least FIG. 6, can be employed in the soft ankle-foot exoskeleton of FIG. 7, for example.

### III. Powered Exoskeleton with Parallel-Elastic Actuation

In various embodiments, the systems and methods can include a 1-DOF bi-directional ankle-foot exoskeleton including a parallel-elastic actuator. The parallel-elastically actuated ankle-foot exoskeleton can provide improvement over certain electro-mechanical actuators. For example, brushless direct current (BLDC) motor advancements have allowed the power and torque density of ankle-foot exoskeletons to increase. However, as the speed of these motors increases, the maximum torque may decrease for a given voltage source. During walking and running gaits, the optimal plantar flexion torque profile of an ankle-foot exoskeleton can occur late in the stance phase of gait, and the peak torque may occur near the time of the peak speed of the ankle. In some cases, the duty cycle of this applied torque may be relatively low, e.g., occurring for around 10% to 40% of the gait cycle.

The parallel-elastic configuration provided herein can leverage various motor technologies to convert electrical energy into mechanical energy that is stored in an elastic element or elements. Such an elastic element or elements can release energy at a higher rate, which may depend on the hysteresis of the elastic material. This effectively decouples the torque and speed capabilities of the actuator, for example. This configuration can change the duty cycle of the motor, such that it is applying torque throughout a larger period of the gait cycle to deflect the elastic elements, thereby allowing (or enabling) slower motor speeds and a less powerful, and therefore smaller, motor to be used.

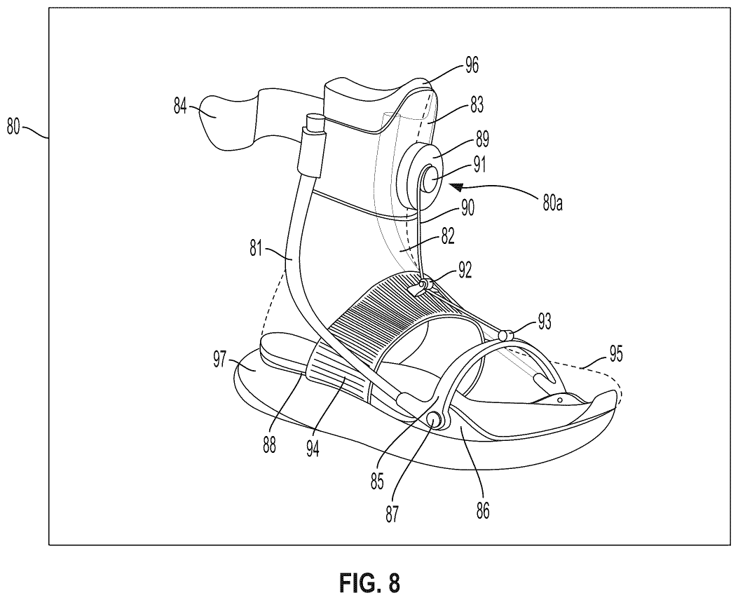

Because of the multi-DOF nature of the human ankle, it can be challenging for the ankle exoskeletons to revolve around spanning this joint without impeding the range-of-motion of the user while also minimizing the movements between the device and the user at the contacting locations, for example. The exoskeletal devices provided by the systems and methods of the following configurations can address the challenges. The components to achieve the exoskeleton characteristics can be discussed in conjunction with at least one of but not limited to FIGS. 8-15, for example.

FIG. 8 illustrates a 1-DOF bi-directional ankle-foot exoskeleton (80) including a parallel-elastic actuator (80a). The exoskeleton (80) can include one or more components, features, or functionalities of at least exoskeleton (1) described in conjunction with FIG. 2, not limited to those discussed herein. The parallel-elastic actuator (80a) can be comprised of lateral (81) and medial (82) parallel-elastic bending beams (e.g., sometimes referred to as lateral beam (81) and medial beam (82), respectively), that may or may not include additional curvatures such as bends or coils. Proximately, the lateral (81) and medial (82) bending beams can be attached to shin guard (83) with calf strap (84). The shin guard (83) with the calf strap (84) can be coupled to at least a portion of the lower limb of the user, such as above at least the ankle of the user and below at least the knee of the user. Distally, the lateral (81) and medial (82) bending beams can be attached to rigid yoke structure (85) spanning the foot from the lateral and medial sides of the foot of the lower limb. The shin guard (83) can include or consist of fabrics and tensile materials that hold the device close to the body of the user during inertial and actuation events, as well as rigid and semi-rigid materials to distribute the force applied by the device and reduce the torsion of the structure around the shank. The yoke structure (85) rotationally can connect to the midfoot aspect (86) of the composite plantar foot plate (88) via a rotational bearing (87).

The parallel-elastic actuator (80a) can be configured with 1-DOF. The parallel-elastic actuator (80 a) (or other actuators discussed herein) may be configured with more than 1-DOF, e.g., 2-DOF. The parallel-elastic actuator (80a) can include at least one motor (89), which may or may not include a mechanical reducer and a clutch. The motor (89) can be attached to a shin guard (83) connecting to a yoke structure (85) via tensile element (90). The parallel-elastic actuator (80a) can include at least one motor idler pulley (91). The motor idler pulley (91) can wind up tensile element, including but not limited to, a belt or cord (e.g., the tensile element (90)), and exert a force on at least the yoke structure (85) via pulley (92) and yoke connecting point (93). Pulley (92) can be mechanically attached to dorsal-arch band (94) to transmit load from the pulley (92) to the plantar carbon foot plate (88). The parallel-elastic actuator (80a) integrates anteriorly with a shoe upper (95) surrounding at least one of a biological foot, ankle, or leg (96). The upper shoe (95) can be attached to a cushioning sole (97) for shock attenuation during gait.

In some configurations, the parallel-elastic actuator (80a) or components of the parallel-elastic actuator (80a) including the motor (89) can be controlled by the control system comprising one or more processors coupled with memory. For example, the control system can actuate or activate the parallel-elastic actuator (80a) into a state of dorsiflexion to augment motion of the user. The state of dorsiflexion can refer to the movement of the foot bringing the toes relatively closer to the shin of the lower limb, for example. The control system can actuate the parallel-elastic actuator (80a) into the dorsiflexion state, for instance, during a portion of the forward movement of the leg detected via one or more sensors discussed herein. In another example, the control system can actuate the parallel-elastic actuator (80a) into a state of plantarflexion to augment motion of the user, for instance, by bringing the toes of the lower limb downward. For example, the control system may actuate the parallel-elastic actuator (80a) into the plantarflexion state during a portion of the backward movement of the leg. The control system can control other operations for the parallel-elastic actuator (80a) or other components of the exoskeleton (80).

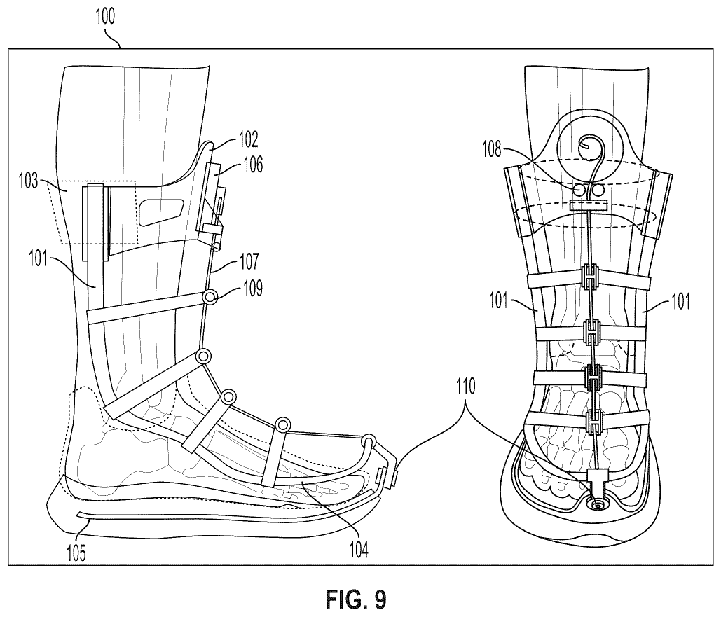

FIG. 9 illustrates a 1-DOF bi-directional ankle-foot exoskeleton (100) including a parallel-elastic ankle actuator (e.g., including features or functionalities similar to the actuator (80a)) with an unpowered subtalar inversion or eversion DOF. Similar to FIG. 8, for instance, the parallel-elastic actuator can be comprised of or include lateral and medial (101) parallel-elastic bending beams. Proximately, the lateral and medial (101) bending beams can be attached to shin guard (102) with calf strap (103). Distally, the lateral and medial (101) bending beams operationally can interface with the ground surface through the distal foot pivot (110) and can be adjacent to the composite plantar foot plate (105) about its midfoot or forefoot region with adequate clearance to rotate freely (104). The beams (101) may not be rigidly attached to the foot plate (105). A motor and motor idler pulley (106), attached to shin guard (102), can connect to tensile element (107) that passes through medial-lateral guidance pulleys (108), through anterior-posterior guidance pulleys (109), to the distal aspect of the plantar foot plate (105) at distal foot pivot (110). Distal pivot (110) can allow a rotational degree of freedom between the lateral and medial (101) parallel-elastic bending beams and the composite plantar foot plate (105) about the distal aspect of the foot plate at (110), such that the biological foot and composite plantar foot plate (105) can rotate about the subtalar joint to enable inversion and eversion movements. In such cases, rotational connector pivot (110) can allow the motor (106) to flex the medial and lateral bending beams (101), storing elastic strain energy, while allowing free movement about the biological subtalar joint.

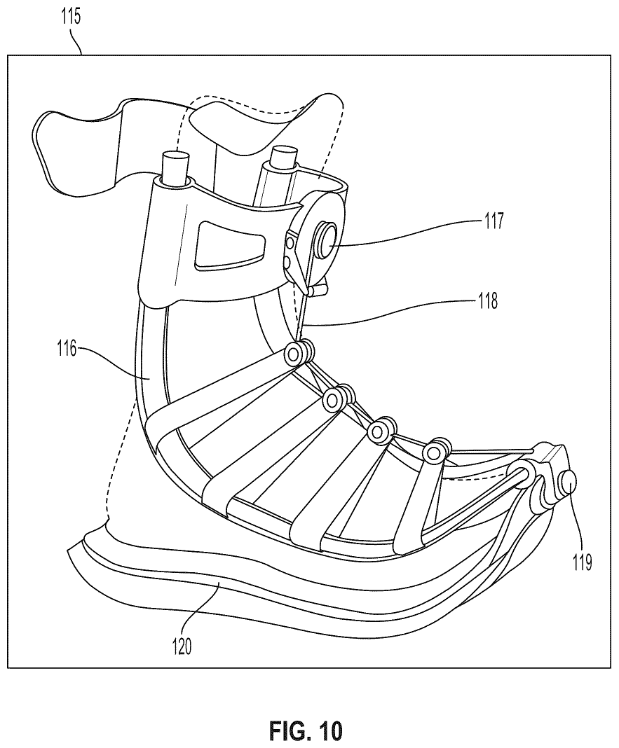

FIG. 10 illustrates the configuration of FIG. 9, where the 1-DOF bi-directional ankle-foot exoskeleton (115) is in a state of dorsiflexion. For example, FIG. 10 shows 1-DOF bi-directional ankle-foot exoskeleton (115) including a parallel-elastic actuator in a state of dorsiflexion. In this case, the medial and lateral bending beams (116) can be operationally coupled to the winch actuator comprising the motor and idler (117) and the tensile element (118) that are engaged in tension. Distally, the lateral and medial (116) bending beams can operationally interface with the ground surface through the distal foot pivot (119) and can be adjacent to the composite plantar foot plate (120) about its midfoot or forefoot region with adequate clearance to rotate the subtalar joint freely. The tensioned winch actuator (117) can result in the foot plate (120) and biological ankle being in a dorsiflexed posture with strain energy stored in the medial and lateral bending beams (116).

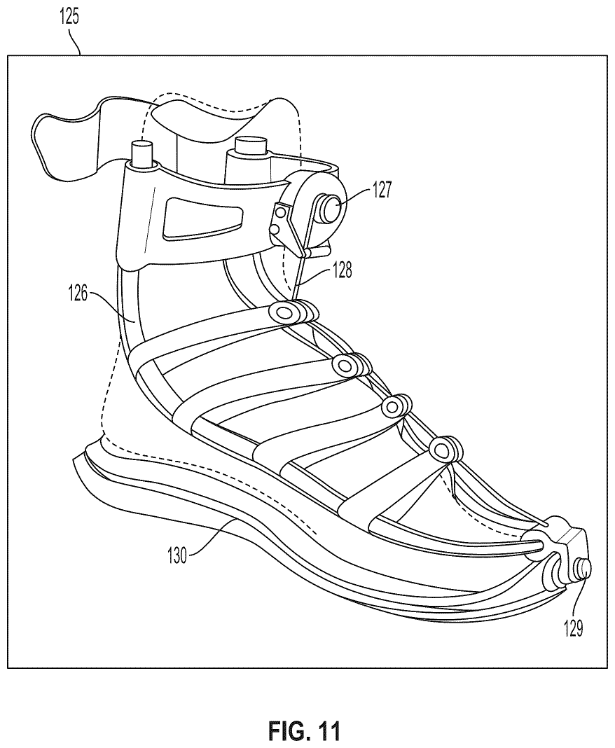

FIG. 11 illustrates 1-DOF bi-directional ankle-foot exoskeleton (125) including a parallel-elastic actuator (127) in a state of plantar flexion. For example, FIG. 11 can include the configuration of FIG. 9, where the 1-DOF bi-directional ankle-foot exoskeleton (125) is in a state of plantar flexion. In this case, the medial and lateral bending beams (126) can be operationally coupled at their proximal aspect to the winch actuator, including the motor and idler (e.g., as part of the parallel-elastic actuator (127)) and the tensile element (128) that are disengaged and not in tension. Distally, the lateral and medial bending beams (126) can operationally interface with the ground surface through the distal foot pivot (129) and can be adjacent to the composite plantar foot plate (130) about its midfoot or forefoot region with adequate clearance to freely rotate about the subtalar joint. The disengaged winch actuator (127) can result in the foot plate (130) and biological ankle being in a plantar flexed posture with no strain energy stored in the medial and lateral bending beams (126).

The operation and control can be discussed herein for various components of the exoskeleton. For example, during operation, the actuator can have multiple distinct modes, such as three modes including but not limited to: zero-torque, powered plantarflexion, and powered dorsiflexion. Zero-torque mode can occur when the actuator is not applying a flexion torque to the ankle. In this mode, the motor (89, 106, 117, 127) can move to a position that corresponds to the position of the human ankle and apply a torque equal and opposite to that torque exerted by the beams (for example, 81, 101, 116, 126). During powered plantar flexion, the motor torque may be less than the torque applied by the beams (81, 101, 116, 126). To modulate the plantar flexion torque, the motor (89, 106, 117, 127) can apply a torque in the opposite direction of the beam. At this point, there may be an energy transfer from the beam (81, 101, 116, 126) to the motor (89, 106, 117, 127). This energy can be converted from the mechanical to the electrical domain and charge the battery of the devices. Powered dorsiflexion can occur when the motor (89, 106, 117, 127) torque exceeds that of the beams (81, 101, 116, 126). This can aid the user during the swing and stance phases of gait, for example.

To apply torque in a way that aids the user (e.g., the movement of the user), a variety of sensors can be integrated into the device. The sensors can include those described in conjunction with at least one of but no limited to FIGS. 2-7. Additionally or alternatively, the sensors can include at least one strain gauge. In some cases, load cells can be incorporated into this device to sense position and torque. The strain gauges installed on or inside the beams (81, 101, 116, 126) in the areas of maximum strain can be used to determine the position of the human ankle. In some cases, a rotary position sensor, such as a joint encoder, could be placed at a distal foot pivot (110, 119, 129) that connects the beams (81, 101, 116, 126) to the foot plate (105, 120, 130) to measure the subtalar inversion/eversion angle. Combining the strain gauge measurements from the medial and lateral beams (81, 101, 116, 126) with the rotary position measurement about the distal foot pivot (110, 119, 129), ankle plantar/dorsiflexion and subtalar inversion/eversion positions can be calculated and used in the control algorithms.

Strain gauges placed on or inside the beams (81, 101, 116, 126) can be used to calculate the torque, which the device is applying to the user. Additionally or alternatively, load cells at the spring attachment points can be used to measure the torque. In addition to a motor, such as in at least FIG. 8 (89), directly coupled to a spool (91), a mechanical reducer can be placed between the spool (91) and motor (89) to amplify the torque of the motor. This reducer can be a planetary gear, spur gear, helical gear, timing belt, or other mechanisms. Such a mechanical reducer can be interspersed between a motor and spool (106, 117, 127) of at least one of but not limited to FIGS. 9-11.

A mechanical clutch can be used to aid the motor (89, 106, 117, 127), for instance, when the mechanism is not moving. This can reduce the electrical energy loss during periods of no movement. For example, at toe-off during walking or running, the motor can rotate and flex the beams (81, 101, 116, 126), dorsiflexing the ankle to achieve foot clearance during the early swing phase. Once the ankle becomes fully dorsiflexed, the mechanical clutch acting on the motor shaft can be engaged under computer control, locking the motor shaft and enabling the motor to turn off. With the motor turned off, no motor torque can be applied, reducing electrical power consumption. Once the foot strikes the ground at the beginning of the next gait cycle, body weight and inertia can (e.g., further) compress the elastic beams (81, 101, 116, 126), as the ankle transitions through controlled dorsiflexion and achieves a state of peak dorsiflexion. Once the beams are (e.g., further) compressed, the tensile element (90, 107, 118, 128) can become slack at which time the clutch can be disengaged to enable the elastic beams (81, 101, 116, 126) to release their stored energy and augment powered plantar flexion during the late stance phase of gait.

In various embodiments, the systems and methods can include 2-DOF bi-directional ankle-foot exoskeleton including differential parallel-elastic actuation. For example, during ambulation, the portions of the biological positive work can occur about the ankle joint. However, the human ankle-foot complex can apply torque about multiple ankle-foot axes resulting in a center-of-pressure trajectory that is curved across the plantar foot surface during gait, such as shown in at least FIG. 4. This effect can be increased on sloped or uneven terrain. The 1-DOF actuator presented the configuration of the parallel-elastic ankle-foot exoskeleton can apply positive work about the ankle joint. This may cause the user to deviate from natural biomechanics during use, for example. To further improve the exoskeleton, the systems and methods can provide a second degree of freedom (e.g., 2-DOF bi-directional ankle-foot exoskeleton including differential parallel-elastic actuation) as discussed herein.

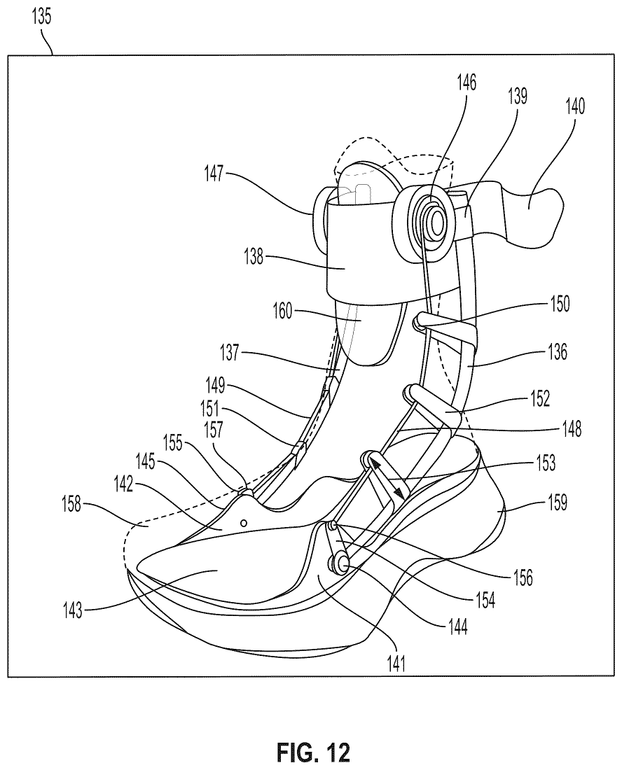

FIG. 12 illustrates a 2-DOF bi-directional ankle-foot exoskeleton (135) including differential parallel-elastic actuators. The differential parallel-elastic actuators can include or be comprised of lateral (136) and medial (137) parallel-elastic bending beams similar to the beams described in conjunction with the aforementioned configuration for 1-DOF bi-directional ankle-foot exoskeleton including a parallel-elastic actuator, for example. Proximately, the lateral (136) and medial (137) bending beams can be attached to a shin guard (138) via attachment means (139). The shin guard (138) can attach to the biological leg using a calf strap (140) and distributes the forces applied by the device and prevents the torsion of the device around the shank. Distally, the lateral (136) and medial (137) bending beams can connect rotationally to the lateral (141) and media (142) tabs of the carbon foot plate (143) via the lateral (144) and medial (145) rotational bearings. A lateral (146) and medial (147) winch actuator with a clutch assembly can be shown in FIG. 12. These assemblies, attached to shin guard (138), can connect to lateral (148) and medial (149) tensile elements that pass through lateral (150) and medial (151) guidance pulleys. The lateral (150) and medial (151) guidance pulleys can be coupled to the lateral (136) and medial (137) parallel-elastic bending beams via offset extensions (152) by an offset distance (153). Distally, the lateral (148) and medial (149) tensile elements can attach to lateral (154) and medial (155) offset brackets via lateral (156) and medial (157) rotational bearings. The differential parallel-elastic actuators can be integrated into a shoe upper (158) about at least one of its lateral, anterior, or medial aspects, among others. The shoe outsole and midsole (159) can offer cushioning to the user of the ankle-foot exoskeleton. Shin load distribution pad (160) can sit beneath shin guard (138) to improve comfort during exoskeleton torque actuation.

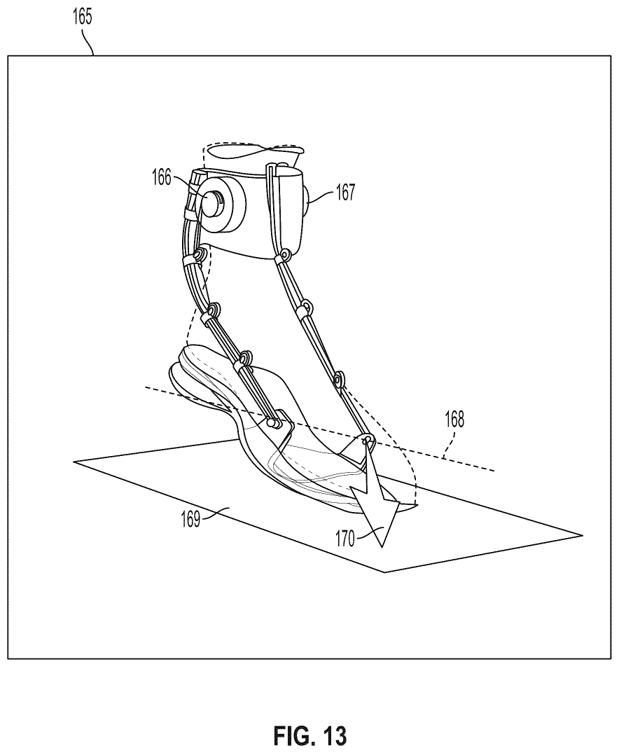

FIG. 13 illustrates the 2-DOF bi-directional ankle-foot exoskeleton (165) including differential parallel-elastic actuation in a state of eversion. For example, FIG. 13 can depict the 2-DOF, bi-directional differential parallel-elastic ankle exoskeleton (135) of at least FIG. 12 in a state of eversion (e.g., exoskeleton (165)). By increasing the tension in the lateral actuator (166), and decreasing the tension in the medial actuator (167), a posture of eversion (168) can be achieved relative to ground plane (169), creating a resulting ground reaction force biased to the medial side (170).

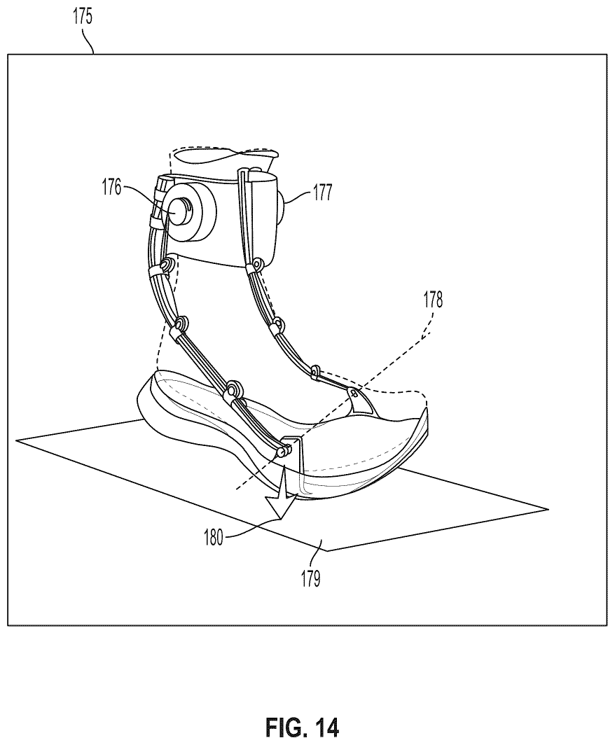

FIG. 14 illustrates a 2-DOF bi-directional ankle-foot exoskeleton (175) including differential parallel-elastic actuation in a state of inversion. For example, FIG. 14 can depict the 2-DOF, bi-directional differential parallel-elastic ankle-foot exoskeleton (135) of at least FIG. 12 in a state of inversion (e.g., exoskeleton (175)). During free-space actuation, by increasing the tension in the medial actuator (177), and decreasing the tension in the lateral actuator (176), a posture of inversion (178) can be achieved. Such an inversion movement about the subtalar joint during the late stance phase of gait can create a center-of-pressure location due to ground reaction forces applied upon the ground surface (179) biased towards the lateral side of the plantar aspect (180) of the foot of the user.

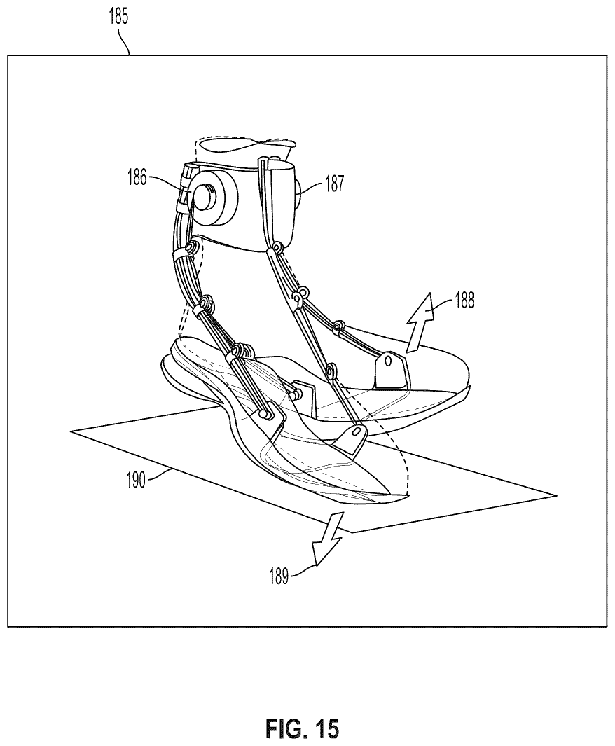

FIG. 15 illustrates a 2-DOF bi-directional ankle-foot exoskeleton (185) including differential parallel-elastic actuation in a state of dorsiflexion and plantar flexion. For example, FIG. 15 can depict the 2-DOF, bi-directional differential parallel-elastic ankle-foot exoskeleton (135) of at least FIG. 12 in a state of dorsiflexion and plantar flexion (e.g., exoskeleton (185)). During free-space actuation, by increasing tension in both the lateral actuator (186) and medial actuator (187), a dorsiflexion ankle posture (188) can be achieved. In this case, symmetric actuator work can flex the lateral and medial bending beams, storing elastic strain energy. In distinction, such as during free-space actuation, by decreasing the tension in both the lateral actuator (186) and medial actuator (187), a plantar flexion ankle posture (189) can be achieved, placing the lateral and medial bending beams in a state of equilibrium. Such dorsi and plantar flexion movements during the stance phase of gait, with the subtalar joint in a neutral position, can create a center-of-pressure location due to ground reaction forces applied upon the ground surface (190) positioned towards the medial-lateral center of the plantar aspect of the foot of the user.

For the operation and control of the configuration for 2-DOF bi-directional ankle-foot exoskeleton including differential parallel-elastic actuation, the bi-directional ankle-foot exoskeleton can leverage a differential mechanism to apply torque to the ankle, which can provide advantages. The control or operations of the exoskeletons discussed herein can be performed on the control system, among other devices or processors. For example, when applying an ankle joint torque, the medial and lateral beams (136, 137) can both apply a positive flexion torque. This can double the ankle torque that a single beam-motor unit can attain. Compared to the configuration for 1-DOF bi-directional ankle-foot exoskeleton including the parallel-elastic actuator, two motors, each with half the torque and power capability, can be used to attain the same actuator ankle torque. The two smaller motors (146, 147) can provide packaging opportunities, as compared to a single large motor, that can result in a lower profile and more convenient form factor.

The 2-DOF nature can allow the control of eversion or inversion torques throughout the gait cycle. This can be used during gait to enable motions closer to natural biomechanics or prescribe a specific motion trajectory. For instance, the actuator can be used to control the plantar-foot center of pressure as the user progresses throughout the stance phase of gait.

In various embodiments, a jointless control configuration or approach can be provided. For example, the powered exoskeleton including a hybrid braided-composite shear interface and parallel-elastic actuation can assist or address devices that do not include traditional artificial rotary joints. The lack of an artificial joint can be advantageous for certain aspects of fit and comfort, however, may present control challenges because a rotary encoder cannot be easily used, for example, to estimate ankle angles. In some cases, the (e.g., intentional) increase in system compliance can present control challenges. To address the control challenges, a combination of sensors, calibrations, and low-latency communication can be implemented.

For example, IMUs can be implemented on various segments of the body of the user to estimate at least one of segment accelerations, velocities, positions, angular velocities, or angles, among other information. Two or more IMUs used on various segments can be synchronized and combined to estimate joint angles. Principle component analysis can be one of many algorithms that can be implemented to estimate joint angles using two or more IMUs, for example.

Because of the compliant nature of the structure, the relative positioning of the IMU to the body may be uncertain or likely to change during use. Both static and dynamic calibration routines can be implemented to estimate relative IMU location and orientation with respect to the body of the user. For example, during a static standing calibration, IMU data can be collected and the average acceleration vector can be used to estimate the vertical axis aligned with gravity. During a walking calibration, the dominant axis of angular rotation can be used to estimate the axis of a swinging shank.

The compliant structure may present challenges to using IMUs while a load is being applied. The force of the actuator may potentially deform the structure and cause the actuator to move relative to the body segment it is attached to, thereby making it potentially difficult to estimate the segment orientation and trajectory. A static calibration routine can be implemented to map the deflection of the actuator as a function of applied torque. Multiple IMUs can be attached to at least one of the actuator or the body segment to measure the relative motion between the two. The relative motion can be used as a force or torque-sensing element.

Multiple sensors and IMUs across multiple body segments may present communication challenges. To synchronize and process the data of these sensors in real-time for control purposes, low-latency communication hardware and software can be implemented. Simple wired connections can be effective, but may present challenges to donning, doffing, or snagging during use. Wireless connections are can be easy to use but may be challenging to reliably implement with a low-latency. The control algorithms can measure and account for the sensor latencies when using these sensors in the control algorithm. Measuring the timing between sent and received packets can provide a latency metric that can be used to determine the accuracy of a data packet. If sensors are experiencing a period of high latency due to damage or interference, the control algorithm can adapt its use accordingly.

Various actuator strategies can be implemented, not limited to those discussed herein. For example, many of the identified configurations may implement linear actuators that wrap a cord or belt to pull on a tensile member. The transmission of these actuators can be increased by implementing a block and tackle mechanism, where the actuated tensile member wraps around a pulley and is affixed to a second member.

The challenge of a shear-inducing jointless exoskeleton can be migration. The actuator forces may potentially cause the structure to migrate along the body segment as force is applied. The braided shear interface can address the challenge by at least one of compressing around the body segment when forces are applied or introducing high-friction elements that prevent relative motion between the braid and the body. To further prevent migration, additional adjustable straps can be added to the braid to introduce a nominal compressive force during a period of zero force. This nominal compressive force and shear friction can prevent the braid from migrating along the body segment due to inertial effects under zero load. The nominal compressive force can be high enough to exert enough friction to counteract the highest expected inertial forces introduced by moving with the actuator.

In some cases, a strategy can include introducing additional connected garments extending across the body to support the braid. In the example of a braid located on a shank, at least one connected garment can be attached to the soft structure that extends across the knee, or across the hip. This soft structure can still allow for natural joint motion while preventing axial migration along the shank. This can be accomplished by adding tensile members along the lines of non-extension, such as over the major joint axes of rotation.

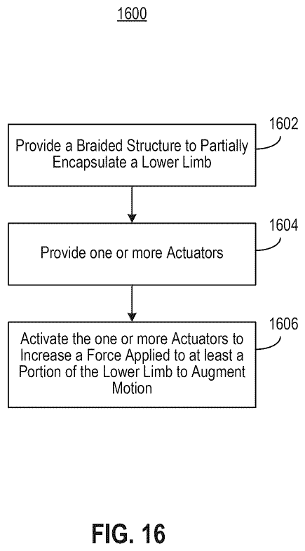

FIG. 16 is an example flow diagram of an example method 1600 for augmenting motion of a user via an ankle-foot exoskeleton. The example method 1600 can be performed or carried out by any devices or components described herein in conjunction with FIGS. 1-15. The method 1600 can include providing a braided structure to partially encapsulate a lower limb, at ACT 1602. At ACT 1604, the method 1600 can include providing one or more actuators. At ACT 1606, the method 1600 can include activating the one or more actuators to increase a force applied to at least a portion of the lower limb to augment motion.

Still referring to FIG. 16, and in further detail, at ACT 1602, a braided structure can be provided for an exoskeleton (e.g. ankle-foot exoskeleton). The braided structure can at least partially encapsulate a lower limb of the user. The braided structure can extend from at least below a knee of the lower limb of the user to at least above an ankle of the lower limb. The braided structure can include a composite shear interface formed of a plurality of materials, e.g., different combinations of materials. In some cases, the braided structure can include a hybrid braided structure. In some other cases, the braided structure can include a hybrid braided-composite shear interface. In some configurations, the braided structure may fully encapsulate a circumference of the lower limb of the user.

At ACT 1604, one or more actuators (e.g., unidirectional actuators or actuator assembly) can be provided for the exoskeleton. The one or more actuators can be mechanically integrated with the braided structure, e.g., via at least one bonding technique or mechanism. The actuator can include one or more components to exert a force on at least a portion of the lower limb of the user, e.g., at least a footplate (e.g., plantar composite footplate) or other components or structures of the exoskeleton coupled to the portion of the lower limb.

At ACT 1606, the exoskeleton can include or be in communication with one or more processors, coupled with memory, to activate the one or more actuators. Activating the one or more actuators can cause the one or more actuators to, for example, increase a force applied to at least a portion of the lower limb to augment motion of the user. The one or more processors can be a part of a computer system or a control system, among other processing components. The one or more processors can control the operations of the one or more actuators, including but not limited to activating or deactivating the actuator(s), increasing or decreasing power output (or force exerted) by the actuator(s), etc.

In some configurations, the exoskeleton can include a plurality of actuators mechanically integrated with the braided structure. The one or more processors can send a signal or command to the actuators to cause the actuators to apply a compression force to the lower limb to augment the motion of the user. The actuators may be configured to monotonically increase a compression force as an actuation force increases to resist the actuation force without slippage via an applied shear force on the lower limb.

The one or more actuators can include a composite plate formed of a braided filament material and a bonding material configured to couple the braided filament material of the composite plate to the braided structure. The bonding material may include an epoxy resin, among other materials. The one or more actuators can include the composite plate that fills in a resin to mount the one or more actuators to the braided structure. The braided structure can include a plurality of layers.

In some arrangements, the braided structure can include a plurality of filaments. At least one of the plurality of filaments can include at least one sensor. The sensor can include, but is not limited to, at least one of joint position sensors, limb translational position sensors, motor position sensors, tensile mechatronic force sensors, plantar-foot pressure sensors, muscle activation sensors, muscle length, velocity, and force sensors, muscle stiffness sensors, skin strain sensors, or other sensing modalities. The sensor can be wearable by the user as part of the exoskeleton or the braided structure. The sensor can modulate, in an updating manner, the ankle plantar flexion torque, for example.

The control system (e.g., one or more processors) can operate the one or more actuators according to measurements from the sensor. For example, the one or more processors can receive data from the sensor. Based on the data, the one or more processors can determine an amount of force to apply to the lower limb of the user to assist or facilitate motion augmentation. The one or more processors can activate the one or more actuators to apply the amount of force in response to the determination.

In some cases, the exoskeleton can include an inertial measurement unit (IMU) configured to determine a shank angle between a shank of the braided structure and a foot of the lower limb. The IMU may be a part of the sensors attached to at least one of the filaments of the braided structure, for example. The one or more processors can determine, based on the shank angle determined via the inertial measurement unit, an ankle angle during actuation of the one or more actuators. The one or more processors can provide commands to the one or more actuators to increase or decrease the force applied to at least the portion of the lower limb according to the ankle angle to augment motion.

In some cases, the exoskeleton can include other components or structures. For example, the exoskeleton can include a shin guard with a calf strap configured to couple to the lower limb of the user. A yoke structure can be provided to the exoskeleton. The yoke structure can be provided at a lateral and medial side of a foot of the lower limb. A parallel-elastic actuator can be provided to the exoskeleton, comprising a lateral beam and a medial beam coupled to the yoke structure. In such configurations, the one or more processors can actuate/activate the parallel-elastic actuator into a state of dorsiflexion to augment motion of the user.

The parallel-elastic actuator is configured with one degree of freedom (1-DOF). In some cases, the parallel-elastic actuator is configured with two degrees of freedom (2-DOF). The parallel-elastic actuator can include at least one of a bend or a coil.

### Further Example Embodiments

The following examples pertain to further embodiments, from which numerous permutations and configurations will be apparent.

Example 1 includes a system for an ankle-foot exoskeleton of a user, comprising: a braided structure configured to at least partially encapsulate a lower limb of the user, the braided structure configured to extend from at least below a knee of the lower limb of the user to at least above an ankle of the lower limb; one or more actuators mechanically integrated with the braided structure; and one or more processors, coupled with memory, to: activate the one or more actuators to cause the one or more actuators to increase a force applied to at least a portion of the lower limb to augment motion of the user.

Example 2 includes the subject matter of Example 1, wherein the braided structure comprises a composite shear interface formed of a plurality of materials.

Example 3 includes the subject matter of any one of Examples 1 and 2, wherein the braided structure comprises a hybrid braided structure.

Example 4 includes the subject matter of any one of Examples 1 through 3, wherein the braided structure comprises a hybrid braided-composite shear interface.

Example 5 includes the subject matter of any one of Examples 1 through 4, wherein the braided structure is configured to fully encapsulate a circumference of the lower limb.

Example 6 includes the subject matter of any one of Examples 1 through 5, comprising: a plurality of actuators mechanically integrated with the braided structure.

Example 7 includes the subject matter of any one of Examples 1 through 6, comprising: the one or more processors to cause the plurality of actuators to apply a compression force to the lower limb to augment the motion of the user.

Example 8 includes the subject matter of any one of Examples 1 through 7, wherein the plurality of actuators are configured to monotonically increase a compression force as an actuation force increases to resist the actuation force without slippage via an applied shear force on the lower limb.

Example 9 includes the subject matter of any one of Examples 1 through 8, comprising: the one or more actuators comprising a composite plate formed of a braided filament material and a bonding material configured to couple the braided filament material of the composite plate to the braided structure.

Example 10 includes the subject matter of any one of Examples 1 through 9, wherein the bonding material comprises an epoxy resin.

Example 11 includes the subject matter of any one of Examples 1 through 10, wherein the one or more actuators comprise the composite plate that fills in a resin to mount the one or more actuators to the braided structure.

Example 12 includes the subject matter of any one of Examples 1 through 11, wherein the braided structure comprises a plurality of layers.

Example 13 includes the subject matter of any one of Examples 1 through 12, comprising: the braided structure comprising a plurality of filaments, wherein at least one of the plurality of filaments comprises a sensor.

Example 14 includes the subject matter of any one of Examples 1 through 13, comprising the one or more processors to: receive data from the sensor; determine an amount of the force to apply based on the data; and activate the one or more actuators to apply the determined amount of force.

Example 15 includes the subject matter of any one of Examples 1 through 14, comprising: an inertial measurement unit configured to determine a shank angle between a shank of the braided structure and a foot of the lower limb.

Example 16 includes the subject matter of any one of Examples 1 through 15, comprising: the one or more processors to determine, based on the shank angle determined via the inertial measurement unit, an ankle angle during actuation of the one or more actuators.

Example 17 includes a method for an ankle-foot exoskeleton of a user, comprising: providing a braided structure configured to at least partially encapsulate a lower limb of the user, the braided structure configured to extend from at least below a knee of the lower limb of the user to at least above an ankle of the lower limb; providing one or more actuators mechanically integrated with the braided structure; and activating, by one or more processors coupled with memory, the one or more actuators to cause the one or more actuators to increase a force applied to at least a portion of the lower limb to augment motion of the user.

Example 18 includes the subject matter of Example 17, wherein the braided structure comprises a composite shear interface formed of a plurality of materials.

Example 19 includes the subject matter of any one of Examples 17 and 18, wherein the braided structure comprises a hybrid braided structure.

Example 20 includes the subject matter of any one of Examples 17 through 19, wherein the braided structure comprises a hybrid braided-composite shear interface.

Example 21 includes the subject matter of any one of Examples 17 through 20, wherein the braided structure is configured to fully encapsulate a circumference of the lower limb.

Example 22 includes the subject matter of any one of Examples 17 through 21, comprising: a plurality of actuators mechanically integrated with the braided structure.

Example 23 includes the subject matter of any one of Examples 17 through 22, comprising: causing, by the one or more processors, the plurality of actuators to apply a compression force to the lower limb to augment the motion of the user.

Example 24 includes the subject matter of any one of Examples 17 through 23, comprising: to monotonically increasing, by the plurality of actuators, a compression force as an actuation force increases to resist the actuation force without slippage via an applied shear force on the lower limb.

Example 25 includes the subject matter of any one of Examples 17 through 24, wherein the one or more actuators comprise a composite plate formed of a braided filament material and a bonding material configured to couple the braided filament material of the composite plate to the braided structure.

Example 26 includes the subject matter of any one of Examples 17 through 25, wherein the bonding material comprises an epoxy resin.

Example 27 includes the subject matter of any one of Examples 17 through 26, wherein the one or more actuators comprise the composite plate that fills in a resin to mount the one or more actuators to the braided structure.

Example 28 includes the subject matter of any one of Examples 17 through 27, wherein the braided structure comprises a plurality of layers.

Example 29 includes the subject matter of any one of Examples 17 through 28, wherein the braided structure comprises a plurality of filaments, wherein at least one of the plurality of filaments comprises a sensor.

Example 30 includes the subject matter of any one of Examples 17 through 29, comprising: receiving, by the one or more processors, data from the sensor; determining, by the one or more processors, an amount of the force to apply based on the data; and activating, by the one or more processors, the one or more actuators to apply the determined amount of force.

Example 31 includes the subject matter of any one of Examples 17 through 30, comprising: determining, by an inertial measurement unit, a shank angle between a shank of the braided structure and a foot of the lower limb.

Example 32 includes the subject matter of any one of Examples 17 through 31, comprising: determining, by the one or more processors, based on the shank angle determined via the inertial measurement unit, an ankle angle during actuation of the one or more actuators.

Example 33 includes a system for an ankle-foot exoskeleton of a user, comprising: a shin guard with a calf strap configured to couple to a lower limb of the user; a yoke structure at a lateral and medial side of a foot of the lower limb; a parallel-elastic actuator comprising a lateral beam and a medial beam coupled to the yoke structure; and one or more processors, coupled with memory, to: actuate the parallel-elastic actuator into a state of dorsiflexion to augment motion of the user.

Example 34 includes the subject matter of Example 33, wherein the parallel-elastic actuator is configured with one degree of freedom.

Example 35 includes the subject matter of any one of Examples 33 and 34, wherein the parallel-elastic actuator is configured with two degrees of freedom.

Example 36 includes the subject matter of any one of Examples 33 through 35, wherein the parallel-elastic actuator comprises at least one of a bend or a coil.

Embodiments of the subject matter and the operations described in this specification can be implemented in digital electronic circuitry, or in computer software, firmware or hardware, including the structures disclosed in this specification and their structural equivalents, or in combinations of one or more of them. The subject matter described in this specification can be implemented as one or more computer programs, e.g., one or more circuits of computer program instructions, encoded on one or more computer storage media for execution by, or to control the operation of, data processing apparatus. Alternatively or in addition, the program instructions can be encoded on an artificially generated propagated signal, e.g., a machine-generated electrical, optical or electromagnetic signal that can be generated to encode information for transmission to suitable receiver apparatus for execution by a data processing apparatus. A computer storage medium can be, or be included in, a computer-readable storage device, a computer-readable storage substrate, a random or serial access memory array or device, or a combination of one or more of them. Moreover, while a computer storage medium may not be a propagated signal, a computer storage medium can be a source or destination of computer program instructions encoded in an artificially generated propagated signal. The computer storage medium can also be, or be included in, one or more separate components or media (e.g., multiple CDs, disks, or other storage devices).

The operations described in this specification can be performed by a data processing apparatus on data stored on one or more computer-readable storage devices or received from other sources. The term “data processing apparatus” or “computing device” encompasses various apparatuses, devices and machines for processing data, including by way of example a programmable processor, a computer, a system on a chip, or multiple ones, or combinations of the foregoing. The apparatus can include special purpose logic circuitry, e.g., an FPGA (field programmable gate array) or an ASIC (application specific integrated circuit). The apparatus can also include, in addition to hardware, code that creates an execution environment for the computer program in question, e.g., code that constitutes processor firmware, a protocol stack, a database management system, an operating system, a cross-platform runtime environment, a virtual machine or a combination of one or more of them. The apparatus and execution environment can realize various different computing model infrastructures, such as web services, distributed computing and grid computing infrastructures.

A computer program (also known as a program, software, software application, script, or code) can be written in any form of programming language, including compiled or interpreted languages, declarative or procedural languages, and it can be deployed in any form, including as a stand-alone program or as a circuit, component, subroutine, object, or other unit suitable for use in a computing environment. A computer program may, but need not, correspond to a file in a file system. A program can be stored in a portion of a file that holds other programs or data (e.g., one or more scripts stored in a markup language document), in a single file dedicated to the program in question, or in multiple coordinated files (e.g., files that store one or more circuits, subprograms, or portions of code). A computer program can be deployed to be executed on one computer or on multiple computers that are located at one site or distributed across multiple sites and interconnected by a communication network.

Processors suitable for the execution of a computer program include, by way of example, microprocessors and any one or more processors of a digital computer. A processor can receive instructions and data from a read only memory or a random access memory or both. The elements of a computer are a processor for performing actions in accordance with instructions and one or more memory devices for storing instructions and data. A computer can include, or be operatively coupled to receive data from or transfer data to, or both, one or more mass storage devices for storing data, e.g., magnetic, magneto optical disks, or optical disks. A computer need not have such devices. Moreover, a computer can be embedded in another device, e.g., a personal digital assistant (PDA), a Global Positioning System (GPS) receiver, or a portable storage device (e.g., a universal serial bus (USB) flash drive), to name just a few. Devices suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto optical disks; and CD ROM and DVD-ROM disks. The processor and the memory can be supplemented by, or incorporated in, special purpose logic circuitry.

To provide for interaction with a user, implementations of the subject matter described in this specification can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech or tactile input.

The implementations described herein can be implemented in any of numerous ways including, for example, using hardware, software or a combination thereof. When implemented in software, the software code can be executed on any suitable processor or collection of processors, whether provided in a single computer or distributed among multiple computers.

Also, a computer may have one or more input and output devices. These devices can be used, among other things, to present a user interface. Examples of output devices that can be used to provide a user interface include printers or display screens for visual presentation of output and speakers or other sound generating devices for audible presentation of output. Examples of input devices that can be used for a user interface include keyboards, and pointing devices, such as mice, touch pads, and digitizing tablets. As another example, a computer may receive input information through speech recognition or in other audible format.

Such computers may be interconnected by one or more networks in any suitable form, including a local area network or a wide area network, such as an enterprise network, and intelligent network (IN) or the Internet. Such networks may be based on any suitable technology and may operate according to any suitable protocol and may include wireless networks, wired networks or fiber optic networks.

A computer employed to implement at least a portion of the functionality described herein may comprise a memory, one or more processing units (also referred to herein simply as “processors”), one or more communication interfaces, one or more display units, and one or more user input devices. The memory may comprise any computer-readable media, and may store computer instructions (also referred to herein as “processor-executable instructions”) for implementing the various functionalities described herein. The processing unit(s) may be used to execute the instructions. The communication interface(s) may be coupled to a wired or wireless network, bus, or other communication means and may therefore allow the computer to transmit communications to or receive communications from other devices. The display unit(s) may be provided, for example, to allow a user to view various information in connection with execution of the instructions. The user input device(s) may be provided, for example, to allow the user to make manual adjustments, make selections, enter data or various other information or interact in any of a variety of manners with the processor during execution of the instructions.

The various methods or processes outlined herein may be coded as software that is executable on one or more processors that employ any one of a variety of operating systems or platforms. Additionally, such software may be written using any of a number of suitable programming languages or programming or scripting tools, and also may be compiled as executable machine language code or intermediate code that is executed on a framework or virtual machine.

In this respect, various inventive concepts may be embodied as a computer readable storage medium (or multiple computer readable storage media) (e.g., a computer memory, one or more floppy discs, compact discs, optical discs, magnetic tapes, flash memories, circuit configurations in Field Programmable Gate Arrays or other semiconductor devices, or other non-transitory medium or tangible computer storage medium) encoded with one or more programs that, when executed on one or more computers or other processors, perform methods that implement the various embodiments of the solution discussed above. The computer readable medium or media can be transportable, such that the program or programs stored thereon can be loaded onto one or more different computers or other processors to implement various aspects of the present solution as discussed above.

The terms “program” or “software” are used herein to refer to any type of computer code or set of computer-executable instructions that can be employed to program a computer or other processor to implement various aspects of embodiments as discussed above. One or more computer programs that when executed perform methods of the present solution need not reside on a single computer or processor, but may be distributed in a modular fashion amongst a number of different computers or processors to implement various aspects of the present solution.

Computer-executable instructions may be in many forms, such as program modules, executed by one or more computers or other devices. Program modules can include routines, programs, objects, components, data structures, or other components that perform particular tasks or implement particular abstract data types. The functionality of the program modules can be combined or distributed as desired in various embodiments.

Also, data structures may be stored in computer-readable media in any suitable form. For simplicity of illustration, data structures may be shown to have fields that are related through location in the data structure. Such relationships may likewise be achieved by assigning storage for the fields with locations in a computer-readable medium that convey relationship between the fields. However, any suitable mechanism may be used to establish a relationship between information in fields of a data structure, including through the use of pointers, tags or other mechanisms that establish relationship between data elements.

Any references to implementations or elements or acts of the systems and methods herein referred to in the singular can include implementations including a plurality of these elements, and any references in plural to any implementation or element or act herein can include implementations including only a single element. References in the singular or plural form are not intended to limit the presently disclosed systems or methods, their components, acts, or elements to single or plural configurations. References to any act or element being based on any information, act or element may include implementations where the act or element is based at least in part on any information, act or element.

Any implementation disclosed herein may be combined with any other implementation, and references to “an implementation,” “some implementations,” “an alternate implementation,” “various implementations,” “one implementation” or the like are not necessarily mutually exclusive and are intended to indicate that a particular feature, structure, or characteristic described in connection with the implementation may be included in at least one implementation. Such terms as used herein are not necessarily all referring to the same implementation. Any implementation may be combined with any other implementation, inclusively or exclusively, in any manner consistent with the aspects and implementations disclosed herein.

References to “or” may be construed as inclusive so that any terms described using “or” may indicate any of a single, more than one and all of the described terms. References to at least one of a conjunctive list of terms may be construed as an inclusive OR to indicate any of a single, more than one and all of the described terms. For example, a reference to “at least one of ‘A’ and ‘B’” can include only ‘A’, only ‘B’, as well as both ‘A’ and ‘B’. Elements other than ‘A’ and ‘B’ can also be included.

The systems and methods described herein may be embodied in other specific forms without departing from the characteristics thereof. The foregoing implementations are illustrative rather than limiting of the described systems and methods.

Where technical features in the drawings, detailed description or any claim are followed by reference signs, the reference signs have been included to increase the intelligibility of the drawings, detailed description, and claims. Accordingly, neither the reference signs nor their absence have any limiting effect on the scope of any claim elements.

The systems and methods described herein may be embodied in other specific forms without departing from the characteristics thereof. The foregoing implementations are illustrative rather than limiting of the described systems and methods. Scope of the systems and methods described herein is thus indicated by the appended claims, rather than the foregoing description, and changes that come within the meaning and range of equivalency of the claims are embraced therein.

1. A system for an ankle-foot exoskeleton of a user, comprising:
a braided structure configured to at least partially encapsulate a lower limb of the user, the braided structure configured to extend from at least below a knee of the lower limb of the user to at least above an ankle of the lower limb;
one or more actuators mechanically integrated with the braided structure; and
one or more processors, coupled with memory, to:
activate the one or more actuators to cause the one or more actuators to increase a force applied to at least a portion of the lower limb to augment motion of the user.

2. The system of claim 1, wherein the braided structure comprises a composite shear interface formed of a plurality of materials.

3. The system of claim 1, wherein the braided structure comprises a hybrid braided structure.

4. The system of claim 1, wherein the braided structure comprises a hybrid braided-composite shear interface.

5. The system of claim 1, wherein the braided structure is configured to fully encapsulate a circumference of the lower limb.

6. The system of claim 1, comprising:
a plurality of actuators mechanically integrated with the braided structure.

7. The system of claim 6, comprising:
the one or more processors to cause the plurality of actuators to apply a compression force to the lower limb to augment the motion of the user.

8. The system of claim 6, wherein the plurality of actuators are configured to monotonically increase a compression force as an actuation force increases to resist the actuation force without slippage via an applied shear force on the lower limb.

9. The system of claim 1, comprising:
the one or more actuators comprising a composite plate formed of a braided filament material and a bonding material configured to couple the braided filament material of the composite plate to the braided structure.

10. The system of claim 9, wherein the bonding material comprises an epoxy resin.

11. The system of claim 9, wherein the one or more actuators comprise the composite plate that fills in a resin to mount the one or more actuators to the braided structure.

12. The system of claim 1, wherein the braided structure comprises a plurality of layers.

13. The system of claim 1, comprising:
the braided structure comprising a plurality of filaments, wherein at least one of the plurality of filaments comprises a sensor.

14. The system of claim 13, comprising the one or more processors to:
receive data from the sensor;
determine an amount of the force to apply based on the data; and
activate the one or more actuators to apply the determined amount of force.

15. The system of claim 1, comprising:
an inertial measurement unit configured to determine a shank angle between a shank of the braided structure and a foot of the lower limb.

16. The system of claim 15, comprising:
the one or more processors to determine, based on the shank angle determined via the inertial measurement unit, an ankle angle during actuation of the one or more actuators.

17. A method for an ankle-foot exoskeleton of a user, comprising:
providing a braided structure configured to at least partially encapsulate a lower limb of the user, the braided structure configured to extend from at least below a knee of the lower limb of the user to at least above an ankle of the lower limb;
providing one or more actuators mechanically integrated with the braided structure; and
activating, by one or more processors coupled with memory, the one or more actuators to cause the one or more actuators to increase a force applied to at least a portion of the lower limb to augment motion of the user.

18. The method of claim 17, wherein the braided structure comprises a composite shear interface formed of a plurality of materials.

19. The method of claim 17, wherein the braided structure comprises a hybrid braided structure.

20. The method of claim 17, wherein the braided structure comprises a hybrid braided-composite shear interface.

21.-36. (canceled)
